(19) United States
(12) Patent Application Publication
(10) Pub. No.: US 2002/0142324 A1
(43) Pub. Date: Oct. 3, 2002

(54) Fungal target genes and methods to identify those genes

(76) Inventors: Xun Wang, San Diego, CA (US); Barbara Gillian Turgeon, San Diego, CA (US); Olen Yoder, San Diego, CA (US); Jianguo Wu, San Diego, CA (US)

Correspondence Address: TORREY MESA RESEARCH INSTITUTE, INTELLECTUAL PROPERTY DEPARTMENT, 3115 MERRYFIELD ROW, SAN DIEGO, CA, 92121, US

(21) Appl. No.: 09/961,527

(22) Filed: Sep. 24, 2001

Related U.S. Application Data
(60) Provisional application No. 60/234,673, filed on Sep. 22, 2000.
(60) Provisional application No. 60/234,650, filed on Sep. 22, 2000.

(51) Int. Cl. C12Q 1/68; C12P 19/34

(52) U.S. Cl. 435/6; 435/91.2; 435/320.1

(57) ABSTRACT

A method for gene identification using genome-wide deletion of genes is provided. The method may be used with any organism capable of homologous recombination, including plants, plant pathogens, microorganisms, and vertebrates. Also provided are genes isolated from Cochliobolus that code for polypeptides essential for normal fungal growth and development and/or for pathogenicity, and methods to identify polypeptides essential to the viability of an organism and/or those associated with pathogenicity. The invention also includes methods of using these polypeptides to identify fungicides. The invention can further be used in a screening assay to identify inhibitors that are potential fungicides.

### CROSS-REFERENCE TO RELATED APPLICATIONS

This application claims the benefit under 35 U.S.C. 119 of U.S. Provisional Patent Application No. 60/234,673 filed Sept. 22, 2000, now abandoned, and U.S. Provisional Patent Application No. 60/234,650 filed Sept. 22, 2000, now abandoned, both of which are herein incorporated by reference in their entirety.

### BACKGROUND

The disciplines traditionally used to investigate the mode of action of fungicides have been biochemistry and physiology. Over the past decade, classical and molecular genetics have been brought to bear on this problem with increasing success. Recently, genetic studies of fungicide resistance have led to advances in the understanding of the site of action of agents active against plant pathogens and, in some cases, to an appreciation of additional mechanisms of resistance to fungicide action.

A number of methods have been developed for the purpose of isolating and disrupting or replacing genes within higher and lower organisms. These methods have proven invaluable for providing information concerning the function of many genes. Once a gene has been isolated and the sequence determined, a transgenic cell or organism can be prepared that expresses or alternatively lacks expression (e.g., a “knockout”) of a particular gene. In order to create such a mutant, a vector is prepared that has sequences having homology to the desired point of insertion in the chromosome of the cell which is generally interrupted by an unrelated sequence, e.g., a marker gene (see, for example, U.S. Pat. Nos. 5,464,764 and 6,100,445). A cell is transformed with the vector and the homologous sequences and the linked unrelated sequences are introduced into the chromosomal DNA through the mechanism of homologous recombination. In lower organisms, such as yeast, Candida albicans genes have been disrupted with PCR products that have 50 to 60 bp of homology to a genomic sequence on each end of a selectable marker (Wilson et al., J. Bacteriol. 181:186801874, 1999). The products were used to disrupt two known genes, ARG5 and ADE2, and two sequences newly identified through the Candida genome project, HRM101 and ENX3. In Dictyostelium discoideum, a mutagenesis technique that used antisense cDNA was employed to identify genes required for development (Spann et al., Proc. Natl. Acad. Sci, USA, 93:5003-5007, 1996). Dictyostelium cells were transformed with a cDNA library made from mRNA of vegetative and developing cells. The cDNA was cloned in an antisense orientation immediately downstream of a vegetative promoter, so that the promoter would drive the synthesis of an antisense RNA transcript. Using this mutagenesis technique, mutants were generated that displayed an identifiable phenotype. The individual cDNA molecules from the mutants were identified and cloned using PCR. When PCR-isolated antisense cDNAs were ligated into an antisense vector and transformed into cells, the phenotypes of the transformed cells matched those of the original mutants from which each cDNA was obtained. Gene disruption transformants were made for three of the novel genes using homologous recombination, in each case generating mutants with phenotypes indistinguishable from those of the original antisense transformants. One disadvantage of such a system is the reliance on the production of an antisense transcript and the requirement that the transcript will inactivate a gene over time.

For higher eukaryotes, a variety of transgenic mammals have been developed. For example, U.S. Pat. No. 4,736,866 describes a mouse containing a transgene encoding an oncogene. U.S. Pat. No. 5,175,384 describes a transgenic mouse deficient in mature T cells. U.S. Pat. No. 5,175,383 describes a mouse with a transgene encoding a gene in the int-2/FGF family. This gene promotes benign prostatic hyperplasia. U.S. Pat. No. 5,175,385 describes a transgenic mouse with enhanced resistance to certain viruses, and WO 92/22645 describes a transgenic mouse deficient in certain lymphoid cell types. Preparation of a knockout mammal requires first introducing a nucleic acid construct that will be used to suppress expression of a particular gene into an undifferentiated cell type termed an embryonic stem (ES) cell. This cell is then injected into a mammalian embryo, where it is integrated into the developing embryo. The embryo is then implanted into a foster mother for the duration of gestation.

Despite the successes which have been achieved using various techniques to alter, e.g., knockout or knockdown, gene function, many of the techniques require that the genes be cloned and that the function of the encoded product is known.

It is generally assumed that most fungicides exert their effect by interacting with a specific protein target molecule. In the past, identification of this target has depended on biochemical and physiological evidence. Because fungicides can often produce effects that are only indirectly linked to the immediate site of action, the determination of direct cause-and-effect relationships can prove very difficult.

Increasingly, researchers are turning to the genetics of fungicide resistance to understand the mechanism of action of a particular chemical or of a class of fungicidal chemicals. Because alterations in resistance most likely at the site of fungicide action, rather than changes in uptake, efflux, or metabolism of the fungicide, it is first necessary to identify a resistant mutant, in which the resistance is due to mutation in a single gene. A gene that confers resistance upon a wild type strain can then, in principle, be isolated using the techniques of fungal DNA transformation. High-efficiency transformation protocols are available in a number of fungi, including several agronomically important plant pathogens (e.g., Alternaria, Cercospora, Cladosporium, Cochliobolus, Colletotrichum, Gaeumannomyces, Magnaporthe, and Ustilago). The availability of DNA sequence databases and the capability to search them rapidly make gene identification increasingly straightforward, at least to the level of protein family by means of motif homology. The final step in identification is to demonstrate that transformation of a wild type strain with a single mutant gene is sufficient to confer resistance.

Studies to elucidate the mode of action of the benzimidazole class of fungicides were the first to utilize classical genetics and later the methods of molecular genetics, using benzimidazole-resistant mutants. At the outset, there was considerable evidence that benzimidazoles, such as benomyl, interfere with fungal cell division and bind to proteins with molecular weights similar to that of tubulin (Davidse et al., in Modem Selective Fungicides, 2^{nd} ed., Jena, New York 1995, p. 305). The analysis of benzimidazole-resistant mutants of Aspergillus demonstrated that resistance could be correlated with changes in benzimidazole binding to tubulin. Gene isolation and sequence analysis then established that resistance to benzimidazoles is due to specific mutations in the gene coding for β-tubulin. The understanding that has emerged from these and subsequent studies is that fungicidal benzimidazoles bind specifically to β-tubulin and inhibit the non-covalent polymerization of α,β-tubulin dimers into stable microtubules (Davidse et at., 1995).

Carboxin is another comparatively old fungicide, with commercial levels of activity, particularly against basidiomycete pathogens. A gene from a carboxin-resistant strain of U. maydis has been cloned, sequenced, and shown to be homologous to known genes encoding the iron-sulfur subunit of succinate dehydrogenase (Keon et al., Curr. Genet., 19:475, 1991). Transformation of wild type strains with this gene was sufficient to confer carboxin resistance. Subsequent comparison of sequences from wild type and resistant strains demonstrated that mutation of two contiguous base pairs, within the codon for a single amino acid of a highly conserved region, was responsible for the resistant phenotype (Broomfield et al., Curr. Genet., 22:117 1992; Keon et al., Biochem. Soc. Trans., 22:234, 1994).

The dicarboximide fungicides are a class with several commercially successful examples that are active against Botrytis cinerea and numerous pathogens affecting vegetable crops. Vinclozolin is one such dicarboximide. To elucidate the mode of action of the dicarboximides in U. maydis, the mechanism of resistance to vinclozolin has been investigated (Orth et al., Phytopathology, 84:1210, 1994). A large number of resistant mutants were isolated, which could be grouped into three complementation groups by subsequent genetic analysis. One of the mutants, U. maydis VR43, carrying resistance gene adr-1, was further characterized (Orth et al., Appl. Environ. Microbiol., 61:2341, 1995). A cosmid DNA library was constructed from this mutant in an autonomously replicating vector and pooled DNA was used for transformation of wild type U. maydis. A 32 kb cosmid conferring resistance to vinclozolin was isolated after four rounds of sib selection. Restriction analysis of the cosmid led to isolation of an 8.7 kb fragment. Sequence analysis of this fragment revealed a 1218 bp open reading frame coding for a serine/threonine protein kinase. Residues essential for kinase catalytic function are conserved within this gene. The role of the protein kinase gene adr-I in conferring resistance was further demonstrated by deleting a 384 bp Narl fragment from the coding region. Transformation of wild type U. maydis with this modified construct did not result in fungicide resistance, confirming the role of the protein kinase gene.

The strobilurin analogs represent the first broad-spectrum class of fungicides since the development of the demethylation inhibitor (DMI) fungicides. Their structure is derived from a series of natural products, particularly strobilurin, oudemansin and myxothiazole, found in certain basidiomycetes and myxobacteria. Aside from somewhat lower activity against the eukaryotic organisms from which some of these natural products are isolated, the strobilurin analogs have remarkable efficacy against a broad range of ascomycetes, basidiomycetes, and oomycetes.

It was recognized early in the study of the original natural products, that these compounds owe their fungicidal activity to inhibition of mitochondrial respiration at the level of complex III (Becker et al., FEBS Lett., 132:329, 1981; Brandt et al., Eur. J. Biochem., 173:499, 1988). Subsequently, a series of experiments was carried out involving yeast mutants resistant to the natural products, in which it was demonstrated that resistance is due to mutations in the mitochondrially encoded gene for apocytochrome b (Di Rago et al., J. Biol. Chem., 264:14543, 1989; Geier et al., Biochem. Soc. Trans., 22:203 1994). More recent data have confirmed that synthetic compounds, designed for optimized fungicidal activity, selectivity and stability, also interact specifically with cytochrome b (Mansfield et al., Biochim. Biophys. Acta, 1015:109 1990).

The phenoxyquinolines, such as LY214352, are a group of compounds with appreciable in vitro activity, although whole-plant disease control is best against Botrytis and Venturia. Although, to date, no development candidate has been announced from this class, it is notable because of the early and successful use of classical and molecular genetics to determine the site of action. In these studies, mutants of A. nidulans resistant to LY214352 were developed (Gustafson et al., Curr. Microbiol., 23:39, 1991), and a cosmid library was prepared from one of them (Gustafson, in Antifungal Agents: Discovery and Mode of Action, Dixon et al., eds., Bios Scientific, Oxford, 1995, p. 111; Gustafson et al., Curr. Genet., 30:159, 1996). A cosmid conferring resistance to a wild type strain was found and sub-cloned to yield an open reading frame with homology to prokaryotic dihydro-orotate dehydrogenase (DHO), an enzyme involved in pyrimidine biosynthesis. Enzyme assays confirmed that the DHO enzymes from the resistant strains had diminished sensitivity to the inhibitors.

Acetyl-CoA carboxylase has long been a target for herbicide design. Several chemical classes are active against this target, with high selectivity for the enzyme from gramineous species. Additionally, an antifungal natural product named soraphen A was isolated from a species of myxobacteria (Gerth et al., J. Antibiot, 47:23, 1994). Experiments in yeast have confirmed that mutants resistant to soraphen A are tightly linked to the accl locus, which codes for acetyl-CoA carboxylase (Vahlensieck et al., Curr. Genet., 25:95, 1994). The ACC1 gene from U. maydis has been cloned (Bailey et al., Mol. Gen. Genet., 249:191, 1995).

Blasticidin is a complex natural product, obtained by fermentation, that is used against rice blast disease caused by Magnaporthe grisea. Even so, a gene that encodes an enzyme catalyzing the deamination of blasticidin has been cloned from Aspergillus terreus isolated from rice paddy soil, and this has been used as a selectable marker for transformation of M. grisea and Schizosaccharomyces pombe (Kimura et al., Mol. Gen. Genet., 242:121, 1994; Kimura et al., Biosci. Biotechnol. Biochem., 56:1177, 1995).

Three examples of anilinopyrimidine fungicides, such as pyrimethanil, are now at or nearing commercialization, with activity against cereal diseases as well as Botrytis and Venturia. A series of studies have shown that these compounds have little effect on conidial germination and germ-tube growth; instead, they appear to inhibit the infection process (summarized in Milling et al., Antifungal Agents: Discovery and Mode of Action, Dixon et al., eds, Bios Scientific, Oxford, 1995, p. 201). Subsequent investigations have demonstrated that the secretion of enzymes involved in the infection process, such as polygalacturonase, pectinase, cellulase, and proteinase, is significantly reduced by fungicide treatment and, furthermore, that the intracellular level of enzymes normally secreted dramatically increases (Miura et al., Pestic. Biochem. Physiol., 48:222, 1994; Milling et al., Pestic. Sci., 45:43, 1995).

The demethylation inhibitor (DMI) group of fungicides comprises a large number of commercially successful compounds, such as triadimenol, which have activity at comparatively low use rates against a wide variety of cereal, vineyard, and orchard pathogens (Kuck et al., Modem Selective Fungicides, 2^{nd} ed., Jena, N.Y., 1995. p. 205). Other analogs are used to treat human and animal mycoses. As a class, these compounds act by inhibiting the cytochrome P450 dependent oxidative demethylation of eburicol in filamentous fungi (or lanosterol in yeasts) in the ergosterol biosynthetic pathway. The bulk of the evidence in support of this site of action was obtained from investigations of the effects of DMI fungicides on the levels of sterol intermediates isolated from treated fungi, from spectral measurement of fungicide binding to cytochrome P450 at physiologically relevant concentrations (Köller, Target Sites of Fungicide Action, CRC Press, Boca Raton, Fla., 1992; Van Den Bossche, in Modem Selective Fungicides 2^{nd} ed., Jena, N.Y., 1995, p. 432), and from studies of the effects of DMI fungicides on ergosterol biosynthesis in cell-free systems (Guan et al., Pest. Biochem. Physiol., 42:262, 1992; Kapteyn, Pestic. Sci., 40:313, 1994).

Several papers have reported the successful cloning and sequencing of lanosterol 14α-demethylase genes from yeast (Kalb et al., Gene, 45:237, 1986; Kalb et al., DNA, 6:529, 1987; Chen et al., Biochem Biophys. Res. Comm. 146:1311, 1987; Chen et al., DNA, 9:617, 1988; Kirsch et al., Gene, 68:229, 1988). The corresponding eburicol 14α-demethylase has been characterized from a filamentous fungus only recently, however (Van Nistelrooy et al., Molec. Gen. Genet., 10:250, 1996). In this work, multiple copies of the gene, isolated from Penicilium italicum, were introduced by transformation into Aspergillus niger. The resulting transformants showed reduced sensitivity to DMI fungicides, indicating that over-expression of the demethylase gene is at least a potential mechanism of resistance. Subsequent analysis of one DMI-resistant laboratory mutant of P. italicum has shown that a point mutation in the demethylase gene is responsible for the resistance phenotype (DeWaard, in Molecular Genetics and Ecology of Pesticide Resistance, American Chemical Society, 1996).

Resistance to DMI fungicides has been documented in a variety of plant-pathogenic fungi (Hollomon, Biochem. Soc. Trans., 21:1047 1993), and cases of a monogenic (Peever et al., Phytopathology, 82:821, 1992) and polygenic (Hollomon, Biochem. Soc. Trans., 21:1047, 1993; Buchenauer in Modem Selective Fungicides: Properties, Applications, Mechanisms of Action, 2^{nd} ed., Jena, N.Y. 1995, p. 259) resistance are known. No examples of target site based resistance have been conclusively proven in strains isolated from the field. Among species of yeast pathogenic in immunocompromised patients, cases of resistance due to gene over-expression and target site based resistance have been recorded (Hitchcock, Biochem Soc. Trans., 21:1039, 1993). A variety of mechanisms of resistance have been encountered in laboratory strains selected upon fungicide challenge with or without mutagenesis. In both yeasts (Buchenauer, 1995; Hitchcock, 1993) and U. maydis (Joseph-Home et al., FEBS Lett., 374:174, 1995; Joseph-Home et al., FEMS Microbiol. Lett., 127:29, 1995), mutant isolates are obtained in which an alteration in the gene encoding sterol Δ5,6-desaturase must have occurred.

There is increasing evidence for the involvement of active efflux mechanisms in DMI fungicide resistance. Early results indicated that, in some DMI-resistant laboratory isolates, resistance could be correlated with levels of fungicide accumulation within fungal cells (De Waard, Pestic. Sci., 22:371, 1988). These results have been extended in other fungi, along with the observation that inhibitors of mitochondrial respiration affect the levels of fungicide accumulation in both sensitive and resistant strains (Stehmann, Pestic Sci. 45:311, 1995). This suggests that energy-dependent efflux mechanisms are already operative in sensitive strains, and perhaps enhanced in resistant ones.

Plasmid membrane proton pumps, often called P-glycoproteins, have been implicated in resistance in human cell lines to a wide variety of anticancer drugs, and increasingly to human antifungals (Hitchcock, Biochem. Soc. Trans. 21:1039, 1993; Monk et al., Crit. Rev. Microbiol., 20:209, 1994). Where this mechanism is operative, pleiotropic resistance to other unrelated inhibitors is often observed. In order to extend the efficacy of traditional chemotherapies, P-glycoproteins are now receiving attention in their own right as targets for inhibition, with the rationale that co-inhibition of the efflux pump may restore or improve the activity of a drug.

A fungicide strategy based on the inhibition of efflux mechanisms has application to plant disease control as well. If fungicide level is, at least in some instances, affected by efflux mechanisms, even in wild-type strains, then combination treatment with an inhibitor of P-glycoprotein action will increase intracellular concentration of the fungicide. Moreover, efflux mechanisms may naturally play a role in pathogenesis mechanisms, both as a means to reduce the intracellular levels of natural plant defense compounds, and to export fungal pathogenesis factors and toxins. If this is correct, then inhibitors of membrane proton pumps themselves may be fungistatic.

While the techniques of molecular genetics have significantly accelerated the rate at which sites of fungicide action can be identified, these methods are laborious and often rely on the generation of resistant mutants. Thus, what is needed is a rapid method to identify genes that encode polypeptides associated with growth, development and/or pathogenicity of pathogens, e.g., fungi.

### SUMMARY OF THE INVENTION

The invention provides a method for the functional analysis of genes, e.g., plant genes or pathogen genes, as such genes of pathogenic fungi. In one embodiment of the invention, a genome-wide deletion strategy is employed, while in another embodiment a genome-wide insertion strategy is employed. For example, a library of genomic DNA or cDNA inserts (DNA fragments) in a vector is contacted with an agent, e.g., an endonuclease such as a restriction enzyme, which causes at least one double strand break in the DNA. The insert size may be relatively small, e.g., at least 100 bp or large, e.g., 50 kb or greater. Preferably, the insert size encompasses at least a portion of the average length of a gene in a particular organism. For example, in Cochliobolus, the average gene is about 1-2 kb in length and is separated from the adjacent gene by about 0.5-1.5. At least one detectable DNA (gene) is introduced into the break site(s) resulting in a library having a detectable DNA which is inserted into a cDNA or genomic DNA fragment, or which replaces a portion of the cDNA or genomic DNA, i.e., the agent causes at least two double strand breaks in the DNA. Any agent causing double strand break(s) may be employed, however, a preferred embodiment of the invention employs a site-specific endonuclease which, for the average size fragment in the library, has at least one recognition site in the fragment for insertion vectors, and, for deletion vectors, at least two recognition sites. The determination of endonuclease recognition site frequency for DNA from any particular organism is within the skill of the art. Thus, for the deletion vectors, the size of the deletion in each unique fragment in the library will vary and be dependent on the agent employed to cause the double strand break. The position of the detectable DNA in the genomic DNA or cDNA insert may be in a coding region or in a non-coding region, e.g., in transcriptional regulatory sequences, centromeres, telomeres and the like, of the DNA fragment. The resulting vectors, preferably containing two regions of homology with genomic DNA in a recipient cell and at least one detectable DNA located between the two regions of homology, are contacted with recipient cells capable of, or which can be induced to undergo, homologous or site-directed recombination. In one embodiment, the homologous sequences and the detectable gene are integrated into the genome by a double crossover event. The resulting gene knockouts or gene insertions can then be screened for a desired phenotype.

Thus, the invention provides a method to prepare a library of modified DNA fragments. The method comprises contacting a library of DNA fragments in a vector with an agent that causes at least one double strand break in at least one fragment to yield a library of DNA fragments having at least one double strand break. Then a detectable polynucleotide or gene is inserted into the double strand break so as to yield a library of modified DNA fragments. The DNA inserts in the library may be cDNAs or genomic DNA fragments. The source of the DNA fragments may DNA or RNA, i.e., cDNA, from any prokaryotic or eukaryotic organism including, but not limited to, microbes, plants, insects, yeast, fungi, or animals including birds, fish and mammals, for example, murine, bovine, canine, equine, caprine, porcine, feline, rat, sheep, rabbits, swine, hamsters, or primate, including human, DNA. Any detectable DNA can be employed in the method of the invention, including but not limited to selectable or screenable marker genes. Any vector may be employed in the practice of the invention, including but not limited to, plasmid, phage, BAC, YAC or cosmid vectors.

Also provided is a library prepared by the method and uses of the library, e.g., to identify genes associated with a particular phenotype. Hence, the invention provides a method of using a library of modified DNA fragments to identify the function of a gene which comprises contacting recipient cells with a library of the invention so as to yield a population of cells comprising at least one recombinant cell in which homologous or site-directed recombination has occurred between the genome of the cell and at least one member of the library. Preferably, the recombinant cell has a detectable phenotype which is associated with the disruption of the corresponding sequence in the genomic DNA of the recombinant cell. Then the recombinant cell is identified and optionally isolated. Once isolated, the gene associated with the phenotype is characterized, e.g., by sequencing. In one embodiment, the DNA fragments are contacted with at least one endonuclease, preferably an endonuclease that does not have a recognition site in the vector, but has at least one recognition site in at least one DNA fragment. Preferably, the source of the recipient cells and the source of the DNA in the library is the same, however, the invention includes the use of a library prepared from a source which is heterologous to the recipient cells. In a preferred embodiment, the recipient cells are those which are capable of, or can be induced to undergo, homologous or site-directed recombination, including but not limited to cells such as plant, insect, yeast, fungi, including fungi of agricultural, industrial, or pharmaceutical importance, or animal cells, e.g., from murine, monkey, bovine, canine, equine, caprine, porcine, feline, rat, sheep, rabbits, fish, birds, swine, hamsters or primates, including undifferentiated cells such as animal and human embryonic stem cells, as well as cultured cells from those cellular sources.

As described herein, saturation mutagenesis of the Cochliobolus heterostrophus genome was accomplished by random deletion of 8-10 kb fragments. For example, a library of 10 kb genomic fragments was constructed and digested with an enzyme having no recognition sites in the vector sequences, allowing most of the fungal insert DNA to be replaced by a selectable drug resistance marker (hygB). Members of the plasmid library were linearized at the vector proximal ends of the fungal sequences, and transformed into a wild type strain of the fungus. Most primary transformants were heterokaryotic and required purification by isolating a single drug resistant conidium. If all conidia are drug sensitive or are shown to carry transforming DNA integrated only at an ectopic position, the mutation may be lethal. All purifiable transformants were then tested for auxotrophy and colony morphology. Prototrophs with normal growth rates were tested for virulence on maize. Mutants with either altered virulence or lethality were noted and the plasmid used for transformation of wild type fungi was sequenced, permitting the deleted DNA to be identified in each case. About 30% of the deletions were lethal, and mutants with altered virulence were found. To more specifically identify the gene(s) responsible for the phenotype of interest, each open reading frame (ORF) affected by the deletion may be targeted individually. The identified genes can be used as potential fungicide targets, or as a means to genetically engineer plants for disease resistance.

A further aspect provides a method for identifying the function of a gene comprising contacting cells with a library constructed as disclosed herein to yield a population of cells containing at least one recombinant cell in which homologous recombination has occurred between the genome of the cell and the modified DNA of at least one member of the library. The recombinant cell is then identified, preferably on the basis of a change in phenotype and the function of the gene determined using the phenotypic change. The recombinant cell can be of any of the types discussed herein, including, but not limited to plant cells, bacterial cells, fungal cells, avian cells and mammalian cells. Also provided is an organism comprising at least one such recombinant cell.

One aspect provides an improved method to identify cells that are transformed with a particular modified DNA fragment. For example, for high throughput screening of individual cells, e.g., spores of a fungus, a population of cells is contacted with a modified DNA fragment comprising at least a screenable marker, e.g., a visibly detectable marker such as green fluorescence protein, and optionally a selectable marker which preferably provides a growth advantage to cells expressing that marker. In one embodiment of the invention, sporulation of the transformed population of cells is induced and the spores subjected to cell sorting. Spores which express a green fluorescence protein are selected and sorted into individual wells. In another embodiment, cells from the transformed population of cells are subjected to cell sorting and individual cells which fluoresce selected.

In one aspect of the invention, genes from fungi, such as Cochliobolus, are identified which are related to pathogenesis. Such genes may be useful to identify novel fungicides. As described hereinbelow, five Cochliobolus genes were identified including a cluster of four closely linked open reading frames, and another from a separate locus. The cluster was associated with virulence and/or pathogenicity, while the separate locus was associated with viability. The first open reading frame in the cluster encoded a polypeptide having structural similarity to a gene encoding versicolorin B synthase, which is involved in biosynthesis of aflatoxin, a potent carcinogen produced by fungal Aspergillis spp (Brown, Proc. Natl. Acad. Sci., 93:1418, 1996). The second open reading frame encoded a polypeptide having structural similarity to cytochrome P450. Interestingly, two cytochrome P450 monooxygenases are required for aflatoxin biosynthesis (Brown et al., 1996; Keller et al., Fungal Genet. Biol., 21:17, 1997). Moreover, all the 25 odd genes for aflatoxin production are clustered in a chromosomal region of 60-70 kb. Thus, the cluster of genes may represent part of a larger gene cluster that controls biosynthesis of a secondary metabolite (small molecule) that is required for or associated with fungal virulence. The gene from the separate locus encodes a polypeptide that is structurally related to the human TRRAP and yeast TRAP-like protein, a protein kinase. Thus, the polypeptide encoded by this locus may be a polypeptide that alters secretion, i.e., the translocation of molecules such as a toxin, alters the activity of other molecules that interact with translocation polypeptides, and/or is associated with polypeptide processing and maturation (see WO 98/50550). Alternatively, or in addition, the polypeptide encoded by this locus may be a transformation/transcription domain-associated protein, and so may be associated with transcription, or in a signaling pathway that is essential for cell function. The gene encoding the fungal TRAP-like polypeptide comprises SEQ ID NO:6, and the four genes in the cluster encode polypeptides comprising SEQ ID NO:7, SEQ ID NO:9, SEQ ID NO:11 and SEQ ID NO:13, which may be essential for fungal growth and development.

An advantage of the present invention is that the newly discovered essential genes provide the basis for identifying a novel fungicidal mode of action which enables one skilled in the art to easily and rapidly discover novel inhibitors of gene products that are useful as fungicides. Thus, the invention also provides isolated genes or gene products from fungi for assay development for inhibitory compounds with fungicidal activity, as agents which inhibit the function or reduce the activity of any of these gene products in fungi are likely to have detrimental effects on fungi, and are potentially good fungicide candidates. The present invention therefore provides methods of using an isolated polypeptide encoded by one or more of the genes of the invention to identify inhibitors thereof, which can then be used as fungicides to suppress the growth of pathogenic fungi. Pathogenic fungi are defined as those capable of colonizing a host and causing disease. Examples of pathogens for the agents identified by the methods of the invention encompass fungal pathogens including plant pathogens such as Septoria tritici, Ashbya gossypii, Stagnospora nodorum, Botrytis cinerea, Fusarium graminearum, Magnaporthe grisea, Cochliobolus heterostrophus, Colletotrichum heterostrophus, Ustilago maydis, Erisyphe graminis, plant pathogenic oomycetes such as Pythium ultimum and Phytophthora infestans, and human pathogens such as Candida albicans and Aspergillus fumigatus, as well as other mycogens.

Also provided herein are nucleotide sequences derived from Cochliobolus. The nucleotide sequences described herein are set forth in SEQ ID NO:1, SEQ ID NO:3, SEQ ID NO:6, SEQ ID NO:8, SEQ ID NO:10, SEQ ID NO:12, SEQ ID NO:14 and the complements thereof. The encoded polypeptides are set forth in SEQ ID NO:5, SEQ ID NO:7, SEQ ID NO:9, SEQ ID NO:11, and SEQ ID NO:13 and any polynucleotides encoding these polypeptides. Also included are nucleotide sequences substantially similar to those set forth in SEQ ID NO:1, SEQ ID NO:3, SEQ ID NO:6, SEQ ID NO: 8, SEQ ID NO:10, SEQ ID NO:12, SEQ ID NO:14, and the complements thereof. The present invention also encompasses polypeptides whose amino acid sequence are substantially similar to the amino acid sequences set forth in SEQ ID NO:5, SEQ ID NO:7, SEQ ID NO:9, SEQ ID NO:11 and SEQ ID NO:13, and any polynucleotides encoding these polypeptides.

Also provided are expression cassettes containing any of the above disclosed polynucleotide sequences as well as recombinant vectors containing such expression cassettes. Further aspects provide recombinant host cells containing such vectors, where the host cells may be bacterial cells, yeast cells, fungal cells, plant cells and animal cells. Organisms, such as plant and animals, containing such host cells are also provided.

The present invention also includes methods of using these gene products as targets, based on the essentiality of the genes for normal fungal growth and development. Thus one aspect provides a method for identifying an agent or agents have anti-fungal activity comprising contacting a fungus with an agent and determining if the agent binds to at least one of SEQ ID NO.5, SEQ ID NO.7, SEQ ID NO.9 SEQ ID NO.11, SEQ ID NO 13, or polypeptides having sequences substantially similar to any of these sequences. The effect of the binding of the agent on the growth, virulence and/or viability of the fungus is then determined. Also provided are anti-fungal agents identified by the method of the present invention. For example, for genes encoding products that are essential for viability or are associated with virulence, agents that bind to or otherwise alter or modulate the activity of that gene product, preferably inactivate or decrease the activity of the gene product, can be identified. In addition, genes that are associated with pathogenicity (virulence), are particularly useful to genetically engineer plants for disease resistance. This would be done by identifying the chemical structure of the virulence factor itself. For example, a gene encoding a product that alters the activity of the fungal gene product, such as by degrading the fungal gene product may be introduced to the genome of a plant so that the plant would now specifically inactivate the gene product, thus preventing disease.

One aspect provides an isolated nucleic acid molecule comprising a prokaryotic or eukaryotic, e.g., plant or fungal, nucleotide sequence which is substantially similar to a Cochliobolus nucleic acid segment, the expression of which is essential for fungal growth and/or development or is associated with pathogenesis. These sequences can be identified by employing the method described herein or by any other method known to the art, e.g., other gene knockout or insertion methods. Preferably, the nucleotide sequence is DNA from a mammal, fungi or plant, either a dicot or a monocot, which encodes a polypeptide that is identical or substantially similar to a Cochliobolus polypeptide comprising any one of SEQ ID NO:5, SEQ ID NO:7, SEQ ID NO:9, SEQ ID NO:11 or SEQ ID NO:13, e.g., those encoded by SEQ ID NO:1, SEQ ID NO:3, SEQ ID NO:6, SEQ ID NO:8, SEQ ID NO:10, SEQ ID NO:12, SEQ ID NO:14, or the complement thereof. The term “substantially similar”, when used herein with respect to a polypeptide means a polypeptide corresponding to a reference polypeptide, wherein the polypeptide has substantially the same structure and function as the reference polypeptide, e.g., where only changes in amino acid sequence are those which do not affect the polypeptide function. When used for a polypeptide or an amino acid sequence, the percentage of identity between the substantially similar and the reference polypeptide or amino acid sequence is at least 65%, 66%, 67%, 68%, 69%, 70%, e.g., 71%, 72%, 73%, 74%, 75%, 76%,77%,78%,79%,80%,81%,82%,83%,84%,85%,86%,87%,88%,89%, and even 90% or more, e.g., 91%, 92%, 93%, 94%, 95%, 96%, 97%, 98%, up to at least 99%, wherein the reference polypeptide is a Cochliobolus polypeptide comprising any one of SEQ ID NO:5, SEQ ID NO:7, SEQ ID NO:9, SEQ ID NO:11 or SEQ ID NO:13, e.g., encoded by SEQ ID NO:1, SEQ ID NO:3, SEQ ID NO:6, SEQ ID NO:8, SEQ ID NO:10, SEQ ID NO:12, SEQ ID NO:14, or the complement thereof. One indication that two polypeptides are substantially similar to each other is that an agent, e.g., an antibody, which specifically binds to one of the polypeptides, specifically binds to the other.

In its broadest sense, the term “substantially similar”, when used herein with respect to a nucleotide sequence or nucleic acid segment, means a nucleotide sequence or segment corresponding to a reference nucleotide sequence or segment, wherein the corresponding sequence encodes a polypeptide having substantially the same structure and function as the polypeptide encoded by the reference nucleotide sequence. The term “substantially similar” is specifically intended to include nucleotide sequences wherein the sequence has been modified to optimize expression in particular cells. The percentage of identity between the substantially similar nucleotide sequence and the reference nucleotide sequence is at least 65%, 66%, 67%, 68%, 69%, 70%, e.g., 71%, 72%, 73%, 74%, 75%, 76%, 77%, 78%, 79%, 80%, 81%, 82%, 83%, 84%, 85%, 86%, 89%, and even 90% or more, e.g., 91%, 92%, 93%, 94%, 95%, 96%, 97%, 98%, up to at least 99%, wherein the reference sequence is any one of SEQ ID NO:1, SEQ ID NO:3, SEQ ID NO:6, SEQ ID NO:8, SEQ ID NO:10, SEQ ID NO:12 or SEQ ID NO:14, or the complement thereof. Sequence comparisons maybe carried out using a Smith-Waterman sequence alignment algorithm (see e.g. Waterman, Introduction to Computational Biology: Maps, Sequences and Genomes, Chapman & Hall, London, 1995, or http://www bto.usc.edu/software/seqaln/index.html). The localS program, version 1.16, is preferably used with following parameters: match: 1, mismatch penalty: 0.33, open-gap penalty: 2, extended-gap penalty: 2. Further, a nucleotide sequence that is “substantially similar” to a reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0,1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

Hence, the isolated nucleic acid molecules of the invention also include the orthologs of the Cochliobolus sequences disclosed herein, i.e., the corresponding nucleic acid molecules in organisms other than Cochliobolus, including, but not limited to, fungi other than Cochliobolus, preferably pathogenic fungi. An “ortholog” is a gene from a different species that encodes a product having the same function as the product encoded by a gene from a reference organism. The encoded ortholog products likely have at least 70% sequence identity to each other. Hence, the invention includes an isolated nucleic acid molecule comprising a nucleotide sequence encoding a polypeptide having at least 70% identity to a polypeptide encoded by one or more of the Cochliobolus sequences. Databases such GenBank may be employed to identify sequences related to the Cochliobolus sequences. Alternatively, recombinant DNA techniques such as hybridization or PCR may be employed to identify sequences related to the Cochliobolus sequences. Fungal orthologs of each of the isolated Cochliobolus genes described herein were identified. For the first open reading frame (ORF) for the gene cluster there was high similarity to sequences in Fusarium graminearum (E value=1e-155), a pathogen of cereals, and Botrytis cinerea (E value=1e-034), a pathogen of many plants, and weak similarity to Ashbya gossypii (E value=1.3), a pathogen of cotton bolls. The Cochliobolus gene in ORF2 of the gene cluster, which likely encodes NTP pyrophosphohydrolase, showed structural similarity to orthologs in Fusarium and Botrytis (the values were: 3e-066 and 3e-079, respectively). ORF3 encoded a Cochliobolus cytochrome P450 that showed similarity to orthologs in Fusarium and Ashbya (the values were 2e-010 and 1e-021 respectively). ORF4 encoded a polypeptide having structural similarity to orthologs in Fusarium (1e-089); Botrytis (1e-104), and Ashbya (4e-079).

Thus, the invention preferably includes an isolated nucleic acid molecule comprising a nucleotide sequence that encodes a polypeptide that is substantially similar to an Cochliobolus polypeptide encoded by a nucleic acid segment having a sequence comprising any one of SEQ ID NO:1,. SEQ ID NO:3, SEQ ID NO:6, SEQ ID NO:8, SEQ ID NO:10, SEQ ID NO:12 or SEQ ID NO:14. Preferably the polypeptide has substantial identity to the Cochliobolus polypeptide, i.e., the polypeptide has at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, and at least 99%, amino acid sequence identity to an Cochliobolus polypeptide encoded by a nucleic acid segment having a sequence comprising any one of SEQ ID NO:1, SEQ ID NO:3, SEQ ID NO:6, SEQ ID NO:8, SEQ ID NO:10, SEQ ID NO:12 or SEQ ID NO:14. The invention also provides anti-sense nucleic acid molecules corresponding to the sequences described herein. Also provided are expression cassettes, e.g., recombinant vectors, and host cells, comprising the nucleic acid molecule of the invention in which the nucleotide sequence is in either sense or antisense orientation.

The nucleic acid molecules of the invention, their encoded polypeptides and compositions thereof, are useful to identify agents that specifically bind to or otherwise alter the activity of the encoded polypeptide. Thus, further aspects include isolated nucleic acid molecules that are essential for the viability of an organism, as well as compositions and methods for identifying inhibitors of those nucleic acid molecules, including inhibitors of the gene product encoded hereby. The compositions include nucleic acid sequences and the amino acid sequences for the polypeptides or partial-length polypeptides encoded thereby which are useful to screen for agents that inhibit those molecules. In another aspect, the isolated nucleic acid molecules are associated with virulence or pathogenicity and so are useful to identify agents that bind to or otherwise alter the activity of the gene product of those nucleic acid molecules. If the agent is one which is encoded by DNA, e.g., a polypeptide, the expression of that DNA in an organism susceptible to the pathogen, e.g., a plant, may provide tolerance or resistance to the organism to the pathogen, preferably by preventing or inhibiting pathogen infection. Methods of the invention involve stably transforming a susceptible organism or cell with one or more of at least a portion of these nucleotide sequences which confer tolerance or resistance operably linked to a promoter capable of driving expression of that nucleotide sequence in the cells of the organism. By “portion” or “fragment”, as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention. By “resistant” is meant an organism, e.g., a plant which exhibits substantially no phenotypic changes as a consequence of infection with the pathogen. By “tolerant” is meant an organism, e.g., a plant which, although it may exhibit some phenotypic changes as a consequence of infection, does not have a substantially decreased reproductive capacity or substantially altered metabolism. For example, the pathogen has a decreased ability to infect the plant, or there are fewer lesions or other symptoms post-infection.

Other uses for the nucleic acid molecules or polypeptides of the invention, include the use of the polypeptide to raise either polyclonal antibodies or monoclonal antibodies, e.g., antibodies which can be employed in diagnostic assays for the presence of the pathogen, and host cells comprising the nucleic acid molecules, e.g., in antisense orientation, or having a deletion in at least a portion of at least one the genes corresponding to the nucleic acid molecules of the invention. Also, given that one of the genes encodes a putative toxin or may be a peptide synthetase (Watanabe, Chem. Biol., 3, 463, 1996) the toxin may be useful in therapy, e.g., as an anti-cancer agent, an antibiotic, or as an immunosuppressant. For the TRAP-like polypeptide, its expression may affect one or more membrane polypeptides, such as those for toxin secretion, e.g., it may translocate one or more members of a class of toxins or molecules that are, at some level, toxic to the host fungal cell. Thus, inhibitors of the TRAP-like polypeptide or its synthesis may specifically inhibit fungal pathogenicity or growth. In addition, this polypeptide or an inhibitor of the activity thereof may be useful as a therapeutic in disorders associated with protein processing and maturation including endocrine, gastrointestinal, and cardiovascular disorders; in inflammation; and in cancers, particularly those involving secretory and gastrointestinal tissues.

The invention also includes recombinant nucleic acid molecules which have been modified so as to comprise codons other than those present in the unmodified sequence. The recombinant nucleic acid molecules of the invention include those in which the modified codons specify amino acids that are the same as those specified by the codons in the unmodified sequence, as well as those that specify different amino acids, i.e., they encode a variant polypeptide having one or more amino acid substitutions relative to the polypeptide encoded by the unmodified sequence.

The invention further includes a nucleotide sequence which is complementary to one (hereinafter “test” sequence) which hybridizes under stringent conditions with the nucleic acid molecules of the invention as well as RNA which is encoded by the nucleic acid molecule. When the hybridization is performed under stringent conditions, either the test or nucleic acid molecule of invention is preferably supported, e.g., on a membrane or DNA chip. Thus, either a denatured test or nucleic acid molecule of the invention is preferably first bound to a support and hybridization is effected for a specified period of time at a temperature of, e.g., between 55 and 70° C., in double strength citrate buffered saline (SC) containing 0.1% SDS followed by rinsing of the support at the same temperature but with a buffer having a reduced SC concentration. Depending upon the degree of stringency required such reduced concentration buffers are typically single strength SC containing 0.1% SDS, half strength SC containing 0.1% SDS and one-tenth strength SC containing 0.1% SDS.

As also described herein, the 5′ regulatory regions, including the promoters, for each of the 5 genes was identified (approximately 2 kb upstream of the start codon). These sequences may be employed to screen for transcription factors, and/or alter the regulation of linked sequences, e.g., in the fungal genome. For example, if the promoter was particularly strong, it could be used to overproduce a molecule of pharmaceutical interest. Spore-specific promoters might be used to express genes only in spores, which are the infectious form of the fungus. A promoter from a gene having early expression in response to an elicitor molecule while the spore is invading the plant could be employed with a resistance-conferring gene to induce the plant to mount a defensive response earlier than usual.

Therefore, also provided is an isolated nucleic acid molecule comprising a nucleotide sequence that directs transcription, e.g., a promoter, or a linked nucleic acid fragment in a host cell, wherein the nucleotide sequence is identical or substantially similar, i.e., has at least 65%, 66%, 67%, 68%, 69%, 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, and 99%, nucleotide sequence identity to a sequence of a promoter from a Cochliobolus gene comprising an open reading frame of any of one of SEQ ID NO:1, SEQ ID NO:3, SEQ ID NO:6, SEQ ID NO:8, SEQ ID NO:10, SEQ ID NO:12 or SEQ ID NO:14, e.g., SEQ ID NOs:15-19. Thus, the invention also includes orthologs of Cochliobolus promoters. The promoter sequence is preferably about 25 to 2000, e.g., 50 to 500 or 100 to 1400, nucleotides in length. Thus, the present invention includes fragments of SEQ ID Nos. 15-19 that comprise a minimal promoter region. In one embodiment of the invention, the isolated nucleic acid molecule comprises a nucleotide sequence which is the promoter region for any one of the open reading frames of SEQ ID NO:6, SEQ ID NO:8, SEQ ID NO:10, SEQ ID NO:12 or SEQ ID NO:14, or is structurally related to the promoter for SEQ ID NO:6, SEQ ID NO:8, SEQ ID NO:10, SEQ ID NO:12 or SEQ ID NO:14, i.e., is an orthologous promoter, and is linked to the open reading frame for a structural gene. Hence, the present invention further provides an expression cassette or a recombinant vector containing the nucleic acid molecule, and the vector may be a plasmid. Such cassettes or vectors, when present in a cell, tissue or organism result in transcription of the linked nucleic acid fragment in the cell, tissue or organism.

The expression cassettes or vectors of the invention may optionally include other regulatory sequences, e.g., transcription terminator sequences, introns and/or enhancers, and may be contained in a host cell. The expression cassette or vector may augment the genome of a cell or may be maintained extrachromosomally.

The present invention further provides a method of augmenting a host genome by contacting cells with an expression cassette or vector of the invention, i.e., one having a nucleotide sequence that directs transcription of a linked nucleic acid fragment in a host cell, wherein the nucleic sequence is from genomic DNA that has at least 65%, and more preferably at least 70%, identity to the sequence of a promoter from a Cochliobolus gene comprising any one of SEQ ID NOs: 6, 8, 10, 12 or 14 so as to yield transformed plant cells; and regenerating the transformed plant cells to provide a differentiated transformed plant, wherein the differentiated transformed plant expresses the linked fragment in the cells of the plant in response to infection. The present invention also provides a plant prepared by the method, progeny and seed thereof.

### BRIEF DESCRIPTION OF THE FIGURES

These and other features, aspects, and advantages of the present invention will become better understood with regard to the following description, appended claims and accompanying figures where:

FIG. 1 shows a schematic representation of the overall strategy for high throughput gene knockout by homologous recombination using fungi as an example.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, public databases and other references cited in this application are herein incorporated by reference in their entirety as if each individual publication, patent, patent application, public database or other reference were specifically and individually indicated to be incorporated by reference.

As used herein, the terms “animal” and “mammal” include human beings.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nuc. Acid. Res., 19:5081, 1991; Ohtsuka et al., J. Biol. Chem., 260:2605, 1985; Rossolini et al., Molec. Cell. Probes.,8:91, 1994). A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single or double-stranded, optionally containing synthetic, non-natural or altered nucleotide bases capable of incorporation into DNA or RNA polymers. The terms “nucleic acid”, “nucleic acid molecule”, “nucleic acid fragment” or “nucleic acid sequence or segment” may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. Alternatively, fragments or portions of a nucleotide sequence that are useful as hybridization probes generally do not encode fragment proteins retaining biological activity. Thus, fragments or portions of a nucleotide sequence may range from at least about 9 nucleotides, about 12 nucleotides, about 20 nucleotides, about 50 nucleotides, about 100 nucleotides or more.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

“Naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

A “marker gene” encodes a selectable or screenable trait.

“Selectable marker” is a gene whose expression in a cell gives the cell a selective advantage. The selective advantage possessed by the cells transformed with the selectable marker gene may be due to their ability to grow in the presence of a negative selective agent, such as an antibiotic or a herbicide, compared to the growth of non-transformed cells. The selective advantage possessed by the transformed cells, compared to non-transformed cells, may also be due to their enhanced or novel capacity to utilize an added compound as a nutrient, growth factor or energy source. Selectable marker gene also refers to a gene or a combination of genes whose expression in a cell gives the cell both a negative and/or a positive selective advantage.

The term “chimeric” refers to any gene or DNA that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, DNA that is either heterologous or homologous to the DNA of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

The terms “protein,” “peptide” and “polypeptide” are used interchangeably herein.

By “variants” is intended substantially similar sequences. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis which encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“DNA shuffling” is a method to introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA preferably encodes a variant polypeptide modified with respect to the polypeptide encoded by the template DNA, and may have an altered biological activity with respect to the polypeptide encoded by the template DNA.

The nucleic acid molecules of the invention can be optimized for enhanced expression in species of interest. For plants, see EPA035472; WO91/16432; Perlak et al., Proc. Acad. Natl. Sci., USA, 88:3324, 1991; and Murray et al., Nuc. Acid. Res., 17:477, 1989. In this manner, the genes or gene fragments can be synthesized utilizing species-preferred codons. See, for example, Campbell and Gowri, Plant Physiol., 92:1, 1990 for a discussion of host-preferred codon usage. Thus, the nucleotide sequences can be optimized for expression in any organism. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, Nature, 370:389, 94; Stemmer, Proc. Natl. Acad. Sci. USA, 91:10747, 1994; Crameri et al., Nature, 391:288, 1997; Moore et al., J. Molec. Biol., 272:336, 1997; Zhang et al., Proc. Natl. Acad. Sci. USA, 94:4504, 1997; Crameri et al., Nature, 391:288, 1998; and U.S. Pat. Nos. 5,605,793 and 5,837,458.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook et al., Molecular Cloning, Cold Spring Harbor, N.Y.: Cold Spring Harbor Laboratory Press (1989).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, typically comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

Such expression cassettes may comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The transcriptional cassette will typically include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. For plants, convenient termination regions are available from the Ti-plasmid of A. tumefaciens, such as the octopine synthase and nopaline synthase termination regions. See also, Guerineau et al., Molec. Gen. Genet., 262:141 1991; Proudfoot, Cell, 64:671, 1991; Sanfacon et al., Genes Devel., 5:141, 1991; Mogen et al., Plant Cell, 2:1261, 1990; Munroe et al., Gene, 91:151, 1990; Ballas et al., Nuc. Acids. Res., 17:7891 1989; Joshi et al., Nuc. Acid. Res., 15:9627, 1987.

An oligonucleotide corresponding to a nucleic acid molecule of the invention may be about 30 or fewer nucleotides in length (e.g., 9, 12, 15, 18, 20, 21 or 24, or any number between 9 and 30). Generally specific primers are upwards of 14 nucleotides in length. For optimum specificity and cost effectiveness, primers of 16-24 nucleotides in length may be preferred. Those skilled in the art are well versed in the design of primers for use processes such as PCR. If required, probing can be done with entire restriction fragments of the gene disclosed herein which may be 100' or even 1000' of nucleotides in length.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

A “functional RNA” refers to an antisense RNA, ribozyme, or other RNA that is not translated but performs some function in a cell.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters. However, some suitable regulatory sequences useful in the present invention will include, but are not limited to constitutive plant promoters, plant tissue-specific promoters, plant development specific promoters, inducible plant promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Molec. Biotechnol., 3:225, 1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell, 1:671, 1989.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

The term “intracellular localization sequence” refers to a nucleotide sequence that encodes an intracellular targeting signal. An “intracellular targeting signal” is an amino acid sequence that is translated in conjunction with a protein and directs it to a particular sub-cellular compartment. “Endoplasmic reticulum (ER) stop transit signal” refers to a carboxy-terminal extension of a polypeptide, which is translated in conjunction with the polypeptide and causes a protein that enters the secretory pathway to be retained in the ER. “ER stop transit sequence” refers to a nucleotide sequence that encodes the ER targeting signal. Other intracellular targeting sequences encode targeting signals active in seeds and/or leaves and vacuolar targeting signals.

“Promoter” refers to a nucleotide sequence, usually upstream (5′) to its coding sequence, which controls the expression of the coding sequence by providing the recognition for RNA polymerase and other factors required for proper transcription.

“Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Constitutive promoter” refers to a promoter that is able to express the gene that it controls in all or nearly all of the plant tissues during all or nearly all developmental stages of the plant.

“Regulated promoter” refers to promoters that direct gene expression not constitutively, but in a temporally- and/or spatially-regulated manner, and include both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al., Biochem. Plants, 15:1, 1989. Since in most cases the exact boundaries of regulatory sequences have not been completely defined, DNA fragments of different lengths may have identical promoter activity. Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysome-inducible systems.

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

“Inducible promoter” refers to those regulated promoters that can be turned on in one or more cell types by an external stimulus, such as a chemical, light, hormone, stress, or a pathogen.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of a polynucleotide, such as an endogenous gene or a transgene, in plants. For example, in the case of antisense constructs, expression may refer to the transcription of the antisense DNA only. In addition, expression refers to the transcription and stable accumulation of sense (mRNA) or functional RNA. Expression may also refer to the production of protein.

“Antisense inhibition” refers to the production of antisense RNA transcripts capable of suppressing the expression of protein from an endogenous gene or a transgene.

“Co-suppression” and “transwitch” each refer to the production of sense RNA transcripts capable of suppressing the expression of identical or substantially similar transgene or endogenous genes (U.S. Pat. No.5,231,020).

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes. (English et al., Plant Cell, 8:179, 1996). Gene silencing includes virus-induced gene silencing (Ruiz et al., Plant Cell, 10:937, 1998).

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, CABIOS, 4:11, 1988; the local homology algorithm of Smith et al., Adv. Appl. Math., 2:482, 1981; the homology alignment algorithm of Needleman and Wunsch, J. Molec. Biol.,48:433, 1970; the search-for-similarity method of Pearson and Lipman, Proc. Natl. Acad. Sci. USA, 85:2444, 1988; the algorithm of Karlin and Altschul, Proc. Natl. Acad. Sci. USA, 87:2264, 1990, modified as in Karlin and Altschul, Proc. Natl. Acad. Sci. USA, 90:5873, 1993.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237, 1988; Higgins et al., CABIOS, 5:151, 1989; Corpet et al., Nuc. Acids Res., 16:10881, 1988; Huang et al., CABIOS, 8:155, 1992; and Pearson et al., Meth. Molec. Biol., 24:307, 1994. The ALIGN program is based on the algorithm of Myers and Miller, supra. The BLAST programs of Altschul et al., J. Molec. Biol., 215:403, 1990, are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences (see, e.g., Karlin & Altschul, Proc. Natl. Acad. Sci. USA, 90:5873, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., Nuc. Acids Res., 25:3389, 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). See http://www.ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a nonconservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch, J. Molec. Biol., 48:433, 1970. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267, 1984; Tₘ 81.5° C. +16.6 (log M) +0.41 (% GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus; Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acids, part I, ch. 2, Elsevier, N.Y., 1993. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2×(or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488, 1985; Kunkel et al., Methods in Enzymol., 154:367, 1987; U.S. Pat. No.4,873,192; Walker and Gaastra, Techniques in Molecular Biology, MacMillan, New York, 1983, and the references cited therein. Guidance as to appropriate amino acid substitutions that do not affect biological activity of the protein of interest may be found in the model of Dayhoff et al., Atlas of Protein Sequence and Structure, Natl. Biomed. Res. Fnd., Washington D.C., 1978. Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass both naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, fungus, mammal or a plant into which a heterologous nucleic acid molecule has been introduced. The nucleic acid molecule can be stably integrated into the genome generally known in the art and are disclosed in Sambrook et al., Molecular Cloning, Cold Spring Harbor Press, 1989. See also Innis et al., PCR Protocols, Academic Press, New York, 1995; and Gelfand, PCR Strategies, Academic Press, 1995; and Innis and Gelfand, PCR Methods Manual, Academic Press, 1999. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic,” plants or calli have been through the transformation process and contain a foreign gene integrated into their chromosome. The term “untransformed” refers to normal plants that have not been through the transformation process.

“Transiently transformed” refers to cells in which transgenes and foreign DNA have been introduced, but not selected for stable maintenance.

“Stably transformed” refers to cells that have been selected and regenerated on a selection media following transformation.

“Transient expression” refers to transgene expression in cells, but not selected for its stable maintenance.

“Genetically stable” and “heritable” refer to chromosomally-integrated genetic elements that are stably maintained in the plant and stably inherited by progeny through successive generations.

“Significant increase” is an increase that is larger than the margin of error inherent in the measurement technique, preferably an increase by about 2-fold or greater.

“Significantly less” means that the decrease is larger than the margin of error inherent in the measurement technique, preferably a decrease by about 2-fold, preferably 5-fold, more preferably 10-fold or greater, e.g., 5- or 10-fold more.

“Enzyme activity” means herein the ability of an enzyme to catalyze the conversion or a substrate into a product. A substrate for the enzyme comprises the natural substrate of the enzyme but also comprises analogues of the natural substrate which can also be converted by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

“Fungicide” is a chemical substance used to kill or suppress the growth of fungal cells.

An “inhibitor” is a chemical substance that causes abnormal growth, e.g., by inactivating the enzymatic activity or a protein such as a biosynthetic enzyme, receptor, signal transduction protein, structural gene product, or transport protein that is essential to the growth or survival, or alters the virulence or pathogenicity, of the fungus. In the context of the instant invention, an inhibitor is a chemical substance that alters the activity encoded by any one of SEQ ID NO:5, SEQ ID NO:7, SEQ ID NO:9, SEQ ID NO:11, or SEQ ID NO:13, or their orthologs.

A “minimal promoter” is a promoter element, particularly a TATA element, that is inactive or that has greatly reduced promoter activity in the absence of upstream activation. In the presence of a suitable transcription factor, the minimal promoter functions to permit transcription.

“Modified or altered activity” means that activity that is different from that which naturally occurs in a fungus (i.e., activity that occurs naturally in the absence of direct or indirect manipulation of such activity by man).

A “substrate” is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

“Tolerance” as used herein is the ability of an organism, e.g., a fungus, to continue essentially normal growth or function when exposed to an inhibitor or fungicide in an amount sufficient to suppress the normal growth or function of native, unmodified fungi.

The present invention provides a method for introducing a modified DNA fragment into a prokaryotic or eukaryotic cell, including, but not limited to, fungi, yeast, plant or animal cells. Thus, the invention provides chimeric or transgenic cells and organisms such as transgenic fungi, plants and animals having defined, and specific, gene alterations. Homologous recombination is a well-studied natural cellular process which results in the scission of two nucleic acid molecules having identical or substantially similar sequences (i.e., “homologous” sequences), and the ligation of the two molecules such that one region of each initially present molecule is now ligated to a region of the other initially present molecule (Watson, J. D., In: Molecular Biology of the Gene, 3rd Ed., W. A. Benjamin, Inc., Menlo Park, Calif. (1977); Sedivy, J. M., Bio-Technol. 6:1192-1196 (1988))

Homologous recombination is, thus, a sequence specific process by which cells can transfer a “region” of DNA from one DNA molecule to another. As used herein, a “region” of DNA is intended to generally refer to any nucleic acid molecule. The region may be of any length from a single base to a substantial fragment of a chromosome. For homologous recombination to occur between two DNA molecules, the molecules must possess a “region of homology” with respect to one another. Such a region of homology must be at least two base pairs long. Two DNA molecules possess such a “region of homology” when one contains a region whose sequence is so similar to a region in the second molecule that homologous recombination can occur. Recombination is catalyzed by enzymes which are naturally present in both prokaryotic and eukaryotic cells. The transfer of a region of DNA may be envisioned as occurring through a multi-step process. If either of the two participant molecules is a circular molecule, then the above recombination event results in the integration of the circular molecule into the other participant.

Importantly, if a particular region is flanked by regions of homology (which may be the same, but are preferably different), then two recombinational events may occur, and result in the exchange of a region of DNA between two DNA molecules. Recombination may be “reciprocal,” and thus results in an exchange of DNA regions between two recombining DNA molecules. Alternatively, it may be “nonreciprocal,” (also referred to as “gene conversion”) and result in both recombining nucleic acid molecules having the same nucleotide sequence. There are no constraints regarding the size or sequence of the region which is exchanged in a two-event recombinational exchange. The frequency of recombination between two DNA molecules may be enhanced by treating the introduced DNA with agents which stimulate recombination. Examples of such agents include trimethylpsoralen, UV light, and the like, which are known to the art.

One approach to producing organisms having defined and specific genetic alterations has used homologous recombination to control the site of integration of an introduced marker gene sequence in tumor cells and in fusions between diploid human fibroblast and tetraploid mouse erythroleukemia cells (Smithies et al., Nature 317:230-234, 1985). This approach was further exploited by Thomas, K. R., and co-workers, who described a general method, known as “gene targeting,” for targeting mutations to a preselected, desired gene sequence of an ES cell in order to produce a transgenic animal (Mansour et al., Nature 336:348-352, 1988; Capecchi Trends Genet. 5:70-76, 1989; Capecchi et al., In: Current Communications in Molecular Biology, Capecchi, M. R. (ed.), Cold Spring Harbor Press, Cold Spring Harbor, N.Y., 1989, pp. 45-52. In order to utilize the “gene targeting” method, the gene of interest must have been previously cloned. The method results in the insertion of a detectable gene into a region of a particular gene of interest. Thus, use of the gene targeting method results in the interruption of the contiguous sequences native of a gene of interest in a native genome.

The modified DNA fragment which is to be introduced into the recipient cell contains a region of homology with a region of the cellular genome. In a preferred embodiment, the DNA fragment will contain two regions of homology with the genome (both chromosomal and episomal) of the recipient cell. These regions of homology will preferably flank a marker gene. The regions of homology may be of any size greater than two bases long. Most preferably, the regions of homology will be greater than 10 bases long. The DNA fragment to be introduced may be single stranded, but is preferably double stranded. The DNA fragment may be introduced to the cell as one or more RNA molecules which may be converted to DNA by reverse transcriptase or by other means. Preferably, the DNA fragment to be introduced will be a double stranded linear DNA molecule. In one embodiment of the invention, a closed covalent circular molecule, having the modified DNA fragment is cleaved, to form a linear molecule. A restriction endonuclease capable of cleaving the vector at least a single site outside of the modified DNA fragment is employed to produce either a blunt end or staggered end linear molecule. Preferably, a restriction endonuclease is employed that releases the modified DNA fragment from the vector sequences.

The invention thus provides a method for introducing the homologous sequences in the vector into the genome of an animal or plant or other organism at a specific chromosomal location. The homologous sequences may differ only slightly from a native gene of the recipient cell (for example, it may contain single or multiple base alterations, insertions or deletions relative to the native gene). Thus, the present invention provides a means for manipulating and modulating gene expression and regulation. After permitting the introduction of the DNA molecule(s), the cells are cultured under conventional conditions, as are known in the art.

In order to facilitate the recovery of those cells which have undergone homologous recombination, a detectable DNA (gene) is employed. Preferably, the detectable DNA is a selectable or screenable marker gene. For the purposes of the present invention, any gene sequence whose presence in a cell permits one to identify and optionally isolate the cell may be employed as a detectable DNA sequence. In one embodiment, the presence of the detectable DNA in a recipient cell is recognized by hybridization, by detection of radiolabelled nucleotides, or by other assays of detection which do not require the expression of the detectable gene. Preferably, such sequences are detected using PCR (Mullis et al., Cold Spring Harbor Symp. Quant. Biol. 51:263-273 1986; Erlich et al., EP 50,424; EP 84,796, EP 258,017, EP 237,362; EP 201,184; U.S. Pat. No.4,683,202; U.S. Pat. No.4,582,788; and U.S. Pat. No.4,683,194). PCR achieves the amplification of a specific nucleic acid sequence using at least one, preferably at least two, oligonucleotide primers complementary to regions of the sequence to be amplified. Extension products incorporating the primers then become templates for subsequent replication steps. PCR provides a method for selectively increasing the concentration of a nucleic acid molecule having a particular sequence even when that molecule has not been previously purified and is present only in a single copy in a particular sample. The method can be used to amplify either single or double stranded DNA.

More preferably, however, the detectable gene sequence will be expressed in the recipient cell, and will result in a selectable phenotype. Examples of such detectable gene sequences include the hprt gene (Littlefield, J. W., Science 145:709-710 1964, a xanthine-guanine phosphoribosyltransferase (gpt) gene, a hyg gene, or an adenosine phosphoribosyltransferase (aprt) gene (Sambrook et al., In: Molecular Cloning A Laboratory Manual, 2nd. Ed., Cold Spring Harbor Laboratory Press, N.Y. 1989, a tk gene (i.e., thymidine kinase gene) and especially the tk gene of herpes simplex virus (Giphart-Gassler et al., Mutat. Res. 214:223-232 1989), the nptII gene (Thomas et al., Cell 51:503-512 1987; Mansour et al., Nature 336:348-352 1988), or other genes which confer resistance to amino acid or nucleoside analogues, or antibiotics, etc. Examples of such genes include gene sequences which encode enzymes such as dihydrofolate reductase (DHFR) enzyme, adenosine deaminase (ADA), asparagine synthetase (AS), hygromycin B phosphotransferase, or a CAD enzyme (carbamyl phosphate synthetase, aspartate transcarbamylase, and dihydroorotase) (Sambrook et al., 1989).

Other such genes include other selectable or screenable markers, depending on whether the marker confers a trait which one can ‘elect’ for by chemical means, i.e., through the use of a selective agent (e.g., a herbicide, antibiotic, or the like), or whether it is simply a trait that one can identify through observation or testing, i.e., by ‘screening’ (e.g., the R-locus trait). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a polypeptide that becomes sequestered in the cell wall, and which polypeptide includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

Elements of the present disclosure are exemplified in detail through the use of particular marker genes. However in light of this disclosure, numerous other possible selectable and/or screenable marker genes will be apparent to those of skill in the art in addition to the one set forth herein below. Therefore, it will be understood that the following discussion is exemplary rather than exhaustive. In light of the techniques disclosed herein and the general recombinant techniques which are known in the art, the present invention renders possible the introduction of any gene, including marker genes, into a recipient cell to generate a transformed plant cell, e.g., a monocot cell.

Possible selectable markers for use in connection with the present invention include, but are not limited to, a neo gene, which codes for kanamycin resistance and can be selected for using kanamycin, G418, a gene encoding resistance to bleomycin, and the like; a bar gene which codes for bialaphos resistance; a gene which encodes an altered EPSP synthase protein thus conferring glyphosate resistance; a nitrilase gene such as bxn from Klebsiella ozaenae which confers resistance to bromoxynil; a mutant acetolactate synthase gene (ALS) which confers resistance to imidazolinone, sulfonylurea or other ALS-inhibiting chemicals (European Patent Application 154,204, 1985); a methotrexate-resistant DHFR gene; a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0 218 571, 1987).

An illustrative embodiment of a selectable marker gene capable of being used in systems to select plant transformants is the genes that encode the enzyme phosphinothricin acetyltransferase, such as the bar gene from Streptomyces hygroscopicus or the pat gene from Streptomyces viridochromogenes (U.S. Pat. No. 5,550,318). The enzyme phosphinothricin acetyltransferase (PAT) inactivates the active ingredient in the herbicide bialaphos, phosphinothricin (PPT). PPT inhibits glutamine synthetase, causing rapid accumulation of ammonia and cell death. The success in using this selective system in conjunction with monocots.

Screenable markers that may be employed include, but are not limited to, a β-glucuronidase or uidA gene (GUS) which encodes an enzyme for which various chromogenic substrates are known; an R-locus gene, which encodes a product that regulates the production of anthocyanin pigments (red color) in plant tissues; a beta-lactamase gene, which encodes an enzyme for which various chromogenic substrates are known (e.g., PADAC, a chromogenic cephalosporin); a xy/E gene which encodes a catechol dioxygenase that can convert chromogenic catechols; an alpha-amylase gene; a tyrosinase gene which encodes an enzyme capable of oxidizing tyrosine to DOPA and dopaquinone which in turn condenses to form the easily detectable compound melanin; a beta-galactosidase gene, which encodes an enzyme for which there are chromogenic substrates; a luciferase (lux) gene, which allows for bioluminescence detection; or an aequorin gene, which may be employed in calcium-sensitive bioluminescence detection, or a green fluorescent protein.

Genes from the maize R gene complex are contemplated to be particularly useful as screenable markers for plants. The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. A gene from the R gene complex was applied to maize transformation, because the expression of this gene in transformed cells does not harm the cells. Thus, an R gene introduced into such cells will cause the expression of a red pigment and, if stably incorporated, can be visually scored as a red sector. If a maize line carries dominant alleles for genes encoding the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively any genotype of maize can be utilized if the C1 and R alleles are introduced together.

A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for populational screening for bioluminescence, such as on tissue culture plates, or even for whole plant screening.

The chimeric or transgenic cells or animals of the present invention are prepared by introducing one or more modified DNA fragments into a precursor pluripotent cell, most preferably an ES cell, or equivalent (Robertson, E. J., In: Current Communications in Molecular Biology, Capecchi, M. R. (ed.), Cold Spring Harbor Press, Cold Spring Harbor, N.Y. (1989), pp. 39-44. The term “precursor” is intended to denote only that the cell is a precursor to the desired (“transfected” or “transformed”) cell. The transfected or transformed cell may be cultured in vitro or in vivo, in a manner known in the art (for ES cells used, to form a chimeric or transgenic animal, see, e.g., Evans et al., Nature 292:154-156, 1981).

The chimeric or transgenic plants of the invention are produced through the regeneration of a plant cell which has received a DNA molecule through the use of the methods disclosed herein. Any plant parts (e.g., pollen, flowers, seeds, leaves, branches, fruit, and the like), cell or tissue which can be regenerated to form a whole differentiated plant can be used in the methods of the invention. Suitable plants include, but are not limited to, cells from plant such as corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers; duckweed (Lemna, see WO 00/07210, which includes members of the family Lemnaceae. There are known four genera and 34 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisuica, L. turionifera, L. valdiviana); genus Spirodela (S. intermedia, S. polyrrhiza, S. punctata); genus Woffia (Wa. angusta, Wa. arrhiza, Wa. australina, Wa. borealis, Wa. brasiliensis, Wa. columbiana, Wa. elongata, Wa. globosa, Wa. microscopica, Wa. neglecta) and genus Wofiella (W1. caudata, W1. denticulata, W1. gladiata, W1. hyalina, W1. lingulata, W1. repunda, W1. rotunda, and W1. neotropica). Any other genera or species of Lemnaceae, if they exist, are also aspects of the present invention. Lemna gibba, Lemna minor, and Lemna miniscula are preferred, with Lemna minor and Lemna miniscula being most preferred. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich, 1986); vegetables including tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pin us contorta), and Monterey pine (Pinus radiata), Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo, Acacia, aneth, artichoke, arugula, blackberry, canola, cilantro, clementines, escarole, eucalyptus, fennel, grapefruit, honey dew, jicama, kiwifiuit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, caluliflower, Brassica, e.g., broccoli, cabbage, brussels sprouts, onion, carrot, leek, beet, broad bean, celery, radish, pumpkin, endive, gourd, garlic, snapbean, spinach, squash, turnip, asparagus, and zucchini and ornamental plants include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia.

Preferred forage and turf grass for use in the methods of the invention include alfalfa, orchard grass, tall fescue, perennial ryegrass, creeping bent grass, and redtop.

Preferably, plants of the present invention are crop plants and in particular cereals (for example, corn, alfalfa, sunflower, rice, Brassica, canola, soybean, barley, soybean, sugarbeet, cotton, safflower, peanut, sorghum, wheat, millet, tobacco, and the like), and even more preferably rice, corn and soybean.

In a preferred embodiment, the host cells are monocot or dicot cells, including, but are not limited to, wheat, corn (maize), rice, oat, barley, millet, rye, rape and alfalfa, as well as asparagus, tomato, egg plant, apple, pear, quince, cherry, apricot, pepper, melon, lettuce, cauliflower, Brassica, e.g., broccoli, cabbage, brussels sprout, sugar beet, sugar cane, sweetcorn, onion, carrot, leek, cucumber, tobacco, aubergine, beet, broad bean, carrot, celery, chicory, cotton, radish, pumpkin, hemp, buckwheat, orchardgrass, creeping bent top, redtop, ryegrass, tobacco, turfgrass, tall fescue, cow pea, endive, gourd, grape, raspberry, chenopodium, blueberry, pineapple, avocado, mango, banana, groundnut, nectarine, papaya, garlic, pea, peach, peanut, pepper, pineapple, plum, potato, safflower, snap bean, spinach, squashes, strawberry, sunflower, sorghum, sweet potato, turnip, watermelon, legumes such as Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo, and the like; and ornamental crops including Impatiens, Begonia, Petunia, Pelargonium, Viola, Cyclamen, Verbena, Vinca, Tagetes, Primula, Saint Paulia, Ageratum, Amaranthus, Anthirrhinum, Aquilegia, Chrysanthemum, Cineraria, Clover, Cosmo, Cowpea, Dahlia, Datura, Delphinium, Gerbera, Gladiolus, Gloxinia, Hippeastrum, Mesembryanthemum, Salpiglossis, Zinnia, and the like. More preferably, the host cells are monocot cells such as maize, rice, wheat, barley, oats, and sorghum, which can be regenerated into a transgenic plant.

Any plant tissue capable of subsequent clonal propagation, whether by organogenesis or embryogenesis, may be transformed with a vector of the present invention. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and hypocotyl meristem).

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are preferred Zea mays tissue sources. Selection of tissue sources for transformation of monocots is described in detail in U.S. Pat. No.6,025,545 and PCT publication WO 95/06128.

For certain plant species, different antibiotic or herbicide selection markers may be preferred. Selection markers used routinely in transformation include the nptII gene which confers resistance to kanamycin and related antibiotics (Messing & Vierra, Gene, 19:252, 1982); the bar gene which confers resistance to the herbicide phosphinothricin (White et al., Nuc. Acids Res., 18:1062 1990, Spencer et al., Theor. Appl. Genet., 79:625, 1990), the hph gene which confers resistance to the antibiotic hygromycin, and the dhfr gene, which confers resistance to methotrexate.

Regeneration protocols for transferred plant parts, cells or tissue are known to the art. The mature plants, grown from the transformed plant cells, are selfed to produce an inbred plant. The inbred plant produces seed containing the introduced modified DNA fragment. These seeds can be grown to produce plants that express this desired gene sequence. Plant parts, progeny and variants, and mutants, of the regenerated plants are also included within the scope of this invention. As used herein, variant describes phenotypic changes that are stable and heritable, including heritable variation that is sexually transmitted to progeny of plants.

In one embodiment, the modified DNA fragment which is to be introduced into recipient cells in accordance with the methods of the present invention will be incorporated into a vector (or a derivative thereof) capable of autonomous replication in a host cell. Preferred prokaryotic vectors include plasmids such as those capable of replication in E. coli such as, for example, pBR322, ColE1, pSCO1, pACYC 184, pi VX. Such plasmids are, for example, disclosed by Maniatis et al. (In: Molecular Cloning. A Laboratory Manual, Cold Spring Harbor Press, Cold Spring Harbor, N.Y. (1982)). Bacillus plasmids include pC194, pC221, pT127, etc. Such plasmids are disclosed by Gryczan, T. (In: The Molecular Biology of the Bacilli, Academic Press, N.Y. (1982), pp. 307-329). Suitable Streptomyces plasmids include pIJ101 (Kendall et al., J. Bacteriol. 169:4177-4183, 1987), and Streptomyces bacteriophages such as phi C31 (Chater et al., In: Sixth International Symposium on Actinomycetales Biology Akademiai Kaido, Budapest, Hungary, 1986, pp. 45-54). Pseudomonas plasmids are reviewed by John et al. (Rev. Infect. Dis. 8:693-704, 1986), and Izaki (Jpn. J. Bacteriol. 33:729-742, 1978). Examples of suitable yeast vectors include the yeast 2-micron circle, the expression plasmids YEP13, YCP and YRP, etc., or their derivatives. Such plasmids are well known in the art (Botstein et al., Miami Wntr. Symp. 19:265-274, 1982; Broach, J. R., In: The Molecular Biology of the Yeast Saccharomyces: Life Cycle and Inheritance, Cold Spring Harbor Laboratory, Cold Spring Harbor, N.Y., p. 445-470, 1981; Broach, Cell 28:203-204, 1982). Examples of vectors which may be used to replicate the DNA molecules in a mammalian host include animal viruses such as bovine papilloma virus, polyoma virus, adenovirus, or SV40 virus. Suitable plant vectors include binary vectors (e.g., see U.S. Pat. No.4,940,838).

The transgenic cells that have the modified DNA fragment both and optionally for pathogen can be assayed for the presence of the detectable DNA and optionally for pathogen phenotype that distinguishes the transgenic cell or organism from the wild type cell or organism. Types of phenotypes may include changes in growth pattern and requirements, sensitivity or resistance to infectious agents or chemical substances, changes in the ability to differentiate or the nature of the differentiation, changes in morphology, changes in response to changes in the environment, e.g., physical changes or chemical changes, changes in response to genetic modifications, and the like. For example, the change in cell phenotype may be the change from normal cell growth to uncontrolled cell growth or from a virulent pathogen to a non- or less virulent pathogen.

Alternatively, the change in cell phenotype may be the change from a normal metabolic state to an abnormal metabolic state. In this case, cells are assayed for their metabolite requirement, such as amino acids, sugars, cofactors, or the like, for growth. Once a group of metabolites has been identified that allows for cell growth, where in the absence of such metabolites the cells do not grow, the metabolites are screened individually to identify which metabolite is assimilable or essential.

Alternatively, the change in cell phenotype may be a change in the structure of the cell. In such a case, cells might be visually inspected under a light or electron microscope. The change in cell phenotype may also be a change in the differentiation program of a cell. The change in cell phenotype may further be a change in the commitment of a cell to a specific differentiation program.

After establishing the presence of the detectable gene and preferably a change in phenotype, the chromosomal region flanking the modified DNA or the corresponding vector having the modified DNA may be identified using PCR with the detectable DNA and/or sequence as a primer for unidirectional PCR, or in conjunction with another primer, for bidirectional PCR. The sequence may then be used to probe a cDNA or genomic library for the locus, so that the region may be isolated and sequenced, or to compare it with sequences in a database, so that related, e.g., contiguous, sequences can be identified. Various techniques may be used for identification of the gene at the locus and the polypeptide expressed by the gene. If desired, the encoded polypeptide may be expressed and optionally isolated, for further characterization.

The method includes the inactivation of both gene copies to determine a change in cell phenotype, or a loss of function, associated with the inactivation of specific alleles of the gene. However, it is not necessary that both alleles of a diploid organism be inactivated to result in a detectable phenotype. Therefore, the invention includes heterozygotes and homozygous for the insertion of modified DNA fragments.

In a preferred embodiment, the polypeptides, including those having substantially similar activities to SEQ ID NO:5, SEQ ID NO:7, SEQ ID NO:9, SEQ ID NO:11 or SEQ ID NO:13, are encoded by nucleotide sequences derived from fungi, e.g., Cochliobolus, preferably from pathogenic fungi, desirably identical or substantially similar to the nucleotide sequences set forth in SEQ ID NO:1, SEQ ID NO:3, SEQ ID NO:6, SEQ ID NO:8, SEQ ID NO:10, or SEQ ID NO:14, or the complement thereof. In yet another embodiment, the polypeptides, including those having substantially similar activities to the SEQ ID NO:5, SEQ ID NO:7, SEQ ID NO:9, SEQ ID NO:11 or SEQ ID NO:13, have amino acid sequences identical or substantially similar to the amino acid sequences set forth in SEQ ID NO:5, SEQ ID NO:7, SEQ ID NO:9, SEQ ID NO:11, or SEQ ID NO:13.

In another preferred embodiment, the present invention describes a method for identifying agents having the ability to inhibit or reduce the activity of any one or more of SEQ ID NO:5, SEQ ID NO:7, SEQ ID NO:9, SEQ ID NO:11, or SEQ ID NO:13 in fungi. Preferably, a transgenic (“knockout”) fungus and/or fungal cell, is obtained which preferably is stably transformed, which comprises a deletion in any of SEQ ID NO:6, SEQ ID NO:8, SEQ ID NO:10, SEQ ID NO:12, or SEQ ID NO:14. Thus, in one embodiment, the gene product encoded by the nucleotide sequence is not expressed, or has reduced or aberrant expression. In another embodiment, the transgenic fungus or cell comprises the corresponding non-deleted sequences linked to a promoter to yield a gene product which is overexpressed. An agent is then contacted with the transgenic fungus and/or cell, and the growth development, virulence or pathogenicity of the transgenic fungus and/or cell is determined relative to the growth, development, or pathogenicity, of the corresponding transgenic fungus and/or cell to which the agent was not applied; or to the corresponding non-transgenic fungus and/or cell.

The invention preferably also provides a method for suppressing the growth of a fungus comprising the step of applying to the fungus an agent identified by the methods of the invention. Normal growth is defined as a growth rate substantially similar to that observed in wild type fungus, preferably greater than at least 50% the growth rate observed in wild type fungus. Normal growth and development may also be defined, when used in relation to filamentous fungi, as normal filament development (including normal septation, normal nuclear migration and distribution), normal sporulation, and normal production of any infection structures (e.g. appressoria). Conversely, suppressed or inhibited growth as used herein is defined as less than 50%, preferably less than 10% or less the growth rate observed in wild type or no growth is macroscopically detected at all or abnormal filament development.

As shown in the examples herein, genes that are essential for normal fungal growth and development or for pathogenicity in Cochliobolus can be identified using gene disruption. Having established the essentiality of certain genes in fungi and having identified the genes encoding these essential activities, the inventors thereby provide an important and sought after tool for new fungicide development.

The present invention discloses the genomic nucleotide sequence of the identified Cochliobolus genes as well as the putative amino acid sequence of the encoded polypeptide. The nucleotide sequence corresponding to the genomic DNA coding region is set forth in SEQ ID NO:1, SEQ ID NO:3, SEQ ID NO:6, SEQ ID NO:8, SEQ ID NO:10, SEQ ID NO:12 and SEQ ID NO:14, and the amino acid sequence encoding the polypeptides is set forth in SEQ ID NO:5, SEQ ID NO:7, SEQ ID NO:9, SEQ ID NO:11, and SEQ ID NO:15. The present invention also encompasses an isolated amino acid sequence derived from a fungus, wherein said amino acid sequence is identical or substantially similar to the amino acid sequence encoded by the nucleotide sequence set forth in SEQ ID NO:1, SEQ ID NO:3, SEQ ID NO:6, SEQ ID NO:8, SEQ ID NO:10, SEQ ID NO:12, and SEQ ID NO:14, preferably wherein said amino acid sequence is substantially similar to SEQ ID NO:5, SEQ ID NO:7, SEQ ID NO:9, SEQ ID NO:11 , and SEQ ID NO:15. For example, using BLASTX (2.0.7) programs with the default settings, notable sequence similarities can be identified.

For recombinant production of the polypeptides of the invention in a host organism, a nucleotide sequence encoding a polypeptide that is substantially similar to SEQ ID NO:5, SEQ ID NO:7, SEQ ID NO:9, SEQ ID NO:11, and SEQ ID NO:13, is inserted into an expression cassette designed for the chosen host and introduced into the host where it is recombinantly produced. For example, SEQ ID NO:6, SEQ ID NO:8, SEQ ID NO:10, SEQ ID NO:12, or SEQ ID NO:14, or nucleotide sequence substantially similar to SEQ ID NO:6, SEQ ID NO:8, SEQ ID NO:10, SEQ ID NO:12, or SEQ ID NO:14, can be used for the recombinant production of a polypeptide of the invention. The choice of specific regulatory sequences such as promoter, signal sequence, 5′ and 3′ untranslated sequences, and enhancer appropriate for the chosen host is within the level of skill of the routine in the art. The resultant molecule, containing the individual elements operably linked in proper reading frame, may be inserted into a vector capable of being transformed into the host cell. Suitable expression vectors and methods for recombinant production of proteins are well known for host organisms such as E. coli, yeast, mammalian, and insect cells (see, e.g., Luckow and Summers, Bio/Technology, 6:47, 1988), and baculovirus expression vectors, e.g., those derived from the genome of Autographica californica nuclear polyhedrosis virus (AcMNPV).

In a preferred embodiment, the nucleotide sequence encoding a polypeptide of the invention is derived from an eukaryote, such as a mammal, a fly, a fungus or a yeast, but is preferably derived from a fungus. In a further preferred embodiment, the nucleotide sequence is identical or substantially similar to the nucleotide sequence set forth in SEQ ID NO:6, SEQ ID NO:8, SEQ ID NO:10, SEQ ID NO:12, or SEQ ID NO:14, or encodes a polypeptide whose amino acid sequence is identical or substantially similar to the amino acid sequence set forth in SEQ ID NO:5, SEQ ID NO:7, SEQ ID NO:9, SEQ ID NO:11, or SEQ ID NO:13. The nucleotide sequence set forth in SEQ ID NO:6, SEQ ID NO:8, SEQ ID NO:10, SEQ ID NO:12, or SEQ ID NO:14 encodes a Cochliobolus polypeptide whose amino acid sequence is set forth in SEQ ID NO:5, SEQ ID NO:7, SEQ ID NO:9, SEQ ID NO:11, or SEQ ID NO:13. Recombinantly produced polypeptide is isolated and purified using a variety of standard techniques. The actual techniques that may be used will vary depending upon the host organism used, whether the polypeptide is designed for secretion, and other such factors familiar to the skilled artisan (see, e.g. chapter 6 of Ausubel et al., Short Protocols in Molecular Biology, 3^{rd} ed., Wiley & Sons, New York, 1994).

Recombinantly produced polypeptides are useful for a variety of purposes. For example, they can be used in in vitro assays in a screen with known fungicidal chemicals, whose target has not been identified, to determine if they inhibit the polypeptides. Such in vitro assays may also be used as more general screens to identify agents that inhibit the polypeptides and that are therefore novel fungicide candidates. Alternatively, recombinantly produced polypeptides are used to elucidate the complex structure of these molecules and to further characterize their association with known inhibitors in order to rationally design new inhibitory fungicides. Nucleotide sequences substantially similar to SEQ ID NO:6, SEQ ID NO:8, SEQ ID NO:10, SEQ ID NO:12, or SEQ ID NO:14, and polypeptides substantially similar to SEQ ID NO:5, SEQ ID NO:7, SEQ ID NO:9, SEQ ID NO:11, or SEQ ID NO:13, from any source, including microbial sources, can be used in the assays exemplified herein. Desirably such nucleotide sequences and polypeptides are derived from pathogenic fungi, e.g., Cochliobolus.

Once a polypeptide has been identified as a potential fungicide target, the next step is to develop an assay that allows screening large number of agents to determine which ones interact with the polypeptide. Although it is straightforward to develop assays for polypeptides of known function, developing assays with polypeptides of unknown function is more difficult. This difficulty can be overcome by using technologies that can detect interactions between a polypeptide and an agent without knowing the biological function of the polypeptide. A short description of three methods is presented, including fluorescence correlation spectroscopy, surface-enhanced laser desorption/ionization, and biacore technologies.

Fluorescence Correlation Spectroscopy (FCS) theory was developed in 1972 but it is only in recent years that the technology to perform FCS became available (Madge et al. Phys. Rev. Lett., 29:705, 1972; Maiti et al., Proc. Natl. Acad. Sci, USA, 94:11753, 1997). FCS measures the average diffusion rate of a fluorescent molecule within a small sample volume. The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant polypeptide with a sequence tag, such as a poly-histidine sequence, inserted at the N or C-terminus. The expression takes place in either E. coli, yeast or insect cells. The polypeptide is purified by chromatography. For example, the poly-histidine tag can be used to bind the expressed protein to a metal chelate column such as Ni2+ chelated on iminodiacetic acid agarose. The polypeptide is then labeled with a fluorescent tag such as carboxytetramethylrhodamine or BODIPY7 (Molecular Probes, Eugene, Oreg.). The polypeptide is then exposed in solution to the potential ligand, and its diffusion rate is determined by FCS using instrumentation available from Carl Zeiss, Inc. (Thomwood, N.Y.). Ligand binding is determined by changes in the diffusion rate of the polypeptide.

Surface-Enhanced Laser Desorption/Ionization (SELDI) was invented by Hutchens and Yip during the late 1980's (Hutchens and Yip, Rapid Comm. Mass Spect., 7:576, 1993). When coupled to a time-of-flight mass spectrometer (TOF), SELDI provides a means to rapidly analyze molecules retained on a chip. It can be applied to ligand polypeptide interaction analysis by covalently binding the target protein on the chip and analyze by MS the small molecules that bind to this polypeptide (Worrall et al., Anal Biochem., 70:750, 1998). In a typical experiment, the target to be analyzed is expressed as described for FCS. The purified polypeptide is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system capable of pipetting the ligands in a sequential manner (autosampler). The chip is then submitted to washes of increasing stringency, for example a series of washes with buffer solutions containing an increasing ionic strength. After each wash, the bound material is analyzed by submitting the chip to SELDI-TOF. Ligands that specifically bind the target will be identified by the stringency of the wash needed to elute them.

Biacore relies on changes in the refractive index at the surface layer upon binding of a ligand to a protein immobilized on the layer. In this system, a collection of small ligands is injected sequentially in a 2-5 μl cell with the immobilized protein. Binding is detected by surface plasmon resonance (SPR) by recording laser light refracting from the surface. In general, the refractive index change for a given change of mass concentration at the surface layer, is practically the same for all polypeptides and peptides, allowing a single method to be applicable for any protein (Liedberg et al., Sensors Actuators, 4:299 1983; Malmquist, Nature, 361:187, 1993). In a typical experiment, the target to be analyzed is expressed as described for FCS. The purified protein is then used in the assay without further preparation. It is bound to the Biacore chip either by utilizing the polyhistidine tag or by other interaction such as ion exchange or hydrophobic interaction. The chip thus prepared is then exposed to the potential ligand via the delivery system incorporated in the instruments sold by Biacore (Uppsala, Sweden) to pipette the ligands in a sequential manner (autosampler). The SPR signal on the chip is recorded and changes in the refractive index indicate an interaction between the immobilized target and the ligand. Analysis of the signal kinetics on rate and off rate allows the discrimination between non-specific and specific interaction.

In one embodiment, a suspected fungicide, for example identified by in vitro screening, is applied to fungi at various concentrations. After application of the suspected fungicide, its effect on the fungus, for example inhibition or suppression of growth and development, or virulence is recorded.

Fungicide resistant polypeptides are also obtained using methods involving in vitro recombination, also called DNA shuffling. By DNA shuffling, mutations, preferably random mutations, are introduced into nucleotide sequences encoding the polypeptides of the invention. DNA shuffling also leads to the recombination and rearrangement of sequences within a coding sequence or to recombination and exchange of sequences between two or more different of genes. These methods allow for the production of millions of mutated coding sequences. The mutated genes, or shuffled genes, are screened for desirable properties, e.g. improved tolerance to fungicides and for mutations that provide broad spectrum tolerance to the different classes of inhibitor chemistry. Such screens are well within the abilities of one skilled in the art.

In a preferred embodiment, a mutagenized gene is formed from at least one template gene, wherein the template gene has been cleaved into double-stranded random fragments of a desired size, and comprising the steps of adding to the resultant population of double-stranded random fragments one or more single or double-stranded oligonucleotides, wherein said oligonucleotides comprise an area of identity and an area of heterology to the double-stranded random fragments; denaturing the resultant mixture of double-stranded random fragments and oligonucleotides into single-stranded fragments; incubating the resultant population of single-stranded fragments with a polymerase under conditions which result in the annealing of said single-stranded fragments at said areas of identity to form pairs of annealed fragments, said areas of identity being sufficient for one member of a pair to prime replication of the other, thereby forming a mutagenized double-stranded polynucleotide; and repeating the second and third steps for at least two further cycles, wherein the resultant mixture in the second step of a further cycle includes the mutagenized double-stranded polynucleotide from the third step of the previous cycle, and the further cycle forms a further mutagenized double-stranded polynucleotide, wherein the mutagenized polynucleotide is a mutated gene encoding a product that has altered activity relative to the product encoded by the template gene. In a preferred embodiment, the concentration of a single species of double-stranded random fragment in the population of double-stranded random fragments is less than 1% by weight of the total DNA. In a further preferred embodiment, the template double-stranded polynucleotide comprises at least about 100 species of polynucleotides. In another preferred embodiment, the size of the double-stranded random fragments is from about 5 bp to 5 kb. In a further preferred embodiment, the fourth step of the method comprises repeating the second and the third steps for at least 10 cycles. Such method is described e.g. in Stemmer et al., Nature, 370:389, 1994, in U.S. Pat. No.5,605,793, U.S. Pat. No.5,811,238, and Crameri et al. Nature, 391:288, 1998, as well as in WO 97/20078, and these references are incorporated herein by reference. In a preferred embodiment, for DNAs encoding polypeptides having domains, e.g., peptide synthetases, the resulting shuffled DNAs may encode a gene product that has altered co-factor requirements, altered substrate specificity and/or produces a different product.

In another preferred embodiment, any combination of two or more different genes are mutagenized in vitro by a staggered extension process (StEP), as described e.g. in Zhao et al., Nature Biotech., 16:258, 1998. The two or more genes are used as templates for PCR amplification with the extension cycles of the PCR reaction preferably carried out at a lower temperature than the optimal polymerization temperature of the polymerase. For example, when a thermostable polymerase with an optimal temperature of approximately 72° C. is used, the temperature for the extension reaction is desirably below 72° C., more desirably below 65° C., preferably below 60° C., more preferably the temperature for the extension reaction is 55° C. Additionally, the duration of the extension reaction of the PCR cycles is desirably shorter than usually carried out in the art, more desirably it is less than 30 seconds, preferably it is less than 15 seconds, more preferably the duration of the extension reaction is 5 seconds. Only a short DNA fragment is polymerized in each extension reaction, allowing template switch of the extension products between the starting DNA molecules after each cycle of denaturation and annealing, thereby generating diversity among the extension products. The optimal number of cycles in the PCR reaction depends on the length of the genes to be mutagenized but desirably over 40 cycles, more desirably over 60 cycles, preferably over 80 cycles are used. Optimal extension conditions and the optimal number of PCR cycles for every combination of genes are determined as described in using procedures well-known in the art. The other parameters for the PCR reaction are essentially the same as commonly used in the art. The primers for the amplification reaction are preferably designed to anneal to DNA sequences located outside of the genes, e.g. to DNA sequences of a vector comprising the genes, whereby the different genes used in the PCR reaction are preferably comprised in separate vectors. The primers desirably anneal to sequences located less than 500 bp away from sequences, preferably less than 200 bp away from the sequences, more preferably less than 120 bp away from the sequences. Preferably, the sequences are surrounded by restriction sites, which are included in the DNA sequence amplified during the PCR reaction, thereby facilitating the cloning of the amplified products into a suitable vector.

In another preferred embodiment, fragments of genes having cohesive ends are produced as described in WO 98/05765. The cohesive ends are produced by ligating a first oligonucleotide corresponding to a part of a gene to a second oligonucleotide not present in the gene or corresponding to a part of the gene not adjoining to the part of the gene corresponding to the first oligonucleotide, wherein the second oligonucleotide contains at least one ribonucleotide. A double-stranded DNA is produced using the first oligonucleotide as template and the second oligonucleotide as primer. The ribonucleotide is cleaved and removed. The nucleotide(s) located 5′ to the ribonucleotide is also removed, resulting in double-stranded fragments having cohesive ends. Such fragments are randomly reassembled by ligation to obtain novel combinations of gene sequences.

Any gene or any combination of genes, or orthologs thereof, can be used for in vitro recombination in the context of the present invention, for example, a gene derived from a fungus, such as, e.g., Cochliobolus, e.g. a gene set forth in SEQ ID NO:6, SEQ ID NO:8, SEQ ID NO:10, SEQ ID NO:12 or SEQ ID NO:14. Whole genes or portions thereof are used in the context of the present invention. The library of mutated genes obtained by the methods described above are cloned into appropriate expression vectors and the resulting vectors are transformed into an appropriate host, for example a fungal cell, an algae like Chlamydomonas, a yeast or a bacteria. Host cells transformed with the vectors comprising the library of mutated genes are cultured on medium that contains inhibitory concentrations of the inhibitor and those colonies that grow in the presence of the inhibitor are selected. Colonies that grow in the presence of normally inhibitory concentrations of inhibitor are picked and purified by repeated restreaking. Their plasmids arc purified and the DNA sequences of cDNA inserts from plasmids that pass this test are then determined.

An assay for identifying a modified gene that is tolerant to an inhibitor may be performed in the same manner as the assay to identify inhibitors of the activity with the following modifications: First, a mutant polypeptide is substituted in one of the reaction mixtures for the wild-type polypeptide of the inhibitor assay. Second, an inhibitor of wild type enzyme is present in both reaction mixtures. Third, mutated activity (activity in the presence of inhibitor and mutated enzyme) and unmutated activity (activity in the presence of inhibitor and wild-type enzyme) are compared to determine whether a significant increase in enzymatic activity is observed in the mutated activity when compared to the unmutated activity. Mutated activity is any measure of activity of the mutated enzyme while in the presence of a suitable substrate and the inhibitor. Unmutated activity is any measure of activity of the wild-type enzyme while in the presence of a suitable substrate and the inhibitor.

In a further embodiment according to the invention, a DNA sequence of the invention may also be used for distinguishing among different species of plant pathogenic fungi and for distinguishing fungal pathogens from other pathogens such as bacteria (Weising et al., in, DNA Fingerprinting in Plants and Fungi, CRC Press, Boca Raton, Fla., 1995,p. 157.

A gene can be incorporated in fungal or bacterial cells using conventional recombinant DNA technology. Generally, this involves inserting a DNA molecule comprising a gene into an expression system to which the DNA molecule is heterologous (i.e., not normally present) using standard cloning procedures known in the art. The vector contains the necessary elements for the transcription and translation of the inserted protein-coding sequences in a fungal cell containing the vector. A large number of vector systems known in the art can be used, such as plasmids (van den Hondel and Punt, in, Applied Molecular Genetics in Fungi, Peberdy et al., eds., Cambridge Univ. Press, 1990, p. 1. The components of the expression system may also be modified to increase expression. For example, truncated sequences, nucleotide substitutions, nucleotide optimization or other modifications may be employed. Expression systems known in the art can be used to transform fungal cells under suitable conditions (Lemke and Peng, in The Mycota, Vol 2, Kuck, ed., Springer-Verlang, Berlin, 1997, p. 109). A DNA molecule comprising a nucleotide sequence of the invention is preferably stably transformed and integrated into the genome of the fungal host cells.

Gene sequences intended for expression in transgenic fungi are first assembled in expression cassettes behind a suitable promoter expressible in fungi (Lang-Hinrichs, in, The Mycota, Vol II, Kuck, ed., Springer-Verlag, Berlin, 1997, p. 141; Jacobs and Stahl, in The Mycota, Vol II, Kuck, ed., Springer-Verlag, Berlin, 1997, p. 155). The expression cassettes may also comprise any further sequences required or selected for the expression of the heterologous DNA sequence. Such sequences include, but are not restricted to, transcription terminators, extraneous sequences to enhance expression such as introns, and sequences intended for the targeting of the gene product to specific organelles and cell compartments. These expression cassettes can then be easily transferred to the fungal transformation vectors as described (Lemke and Peng, 1997).

### EXAMPLES

The following examples are intended to provide illustrations of the application of the present invention. The following examples are not intended to completely define or otherwise limit the scope of the invention.

### Example 1

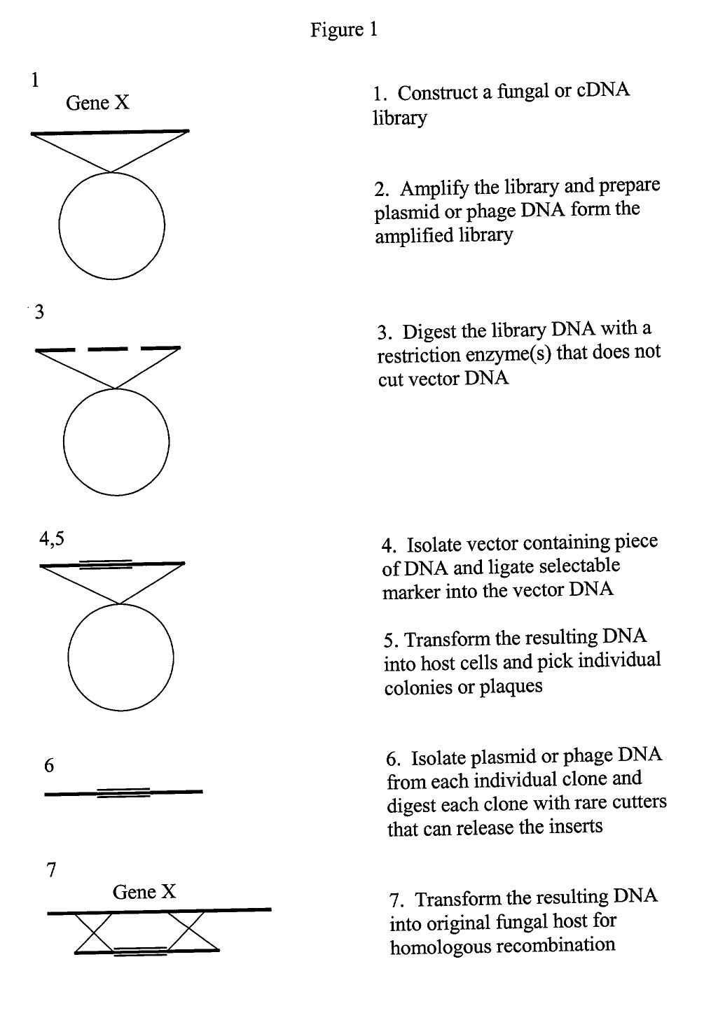

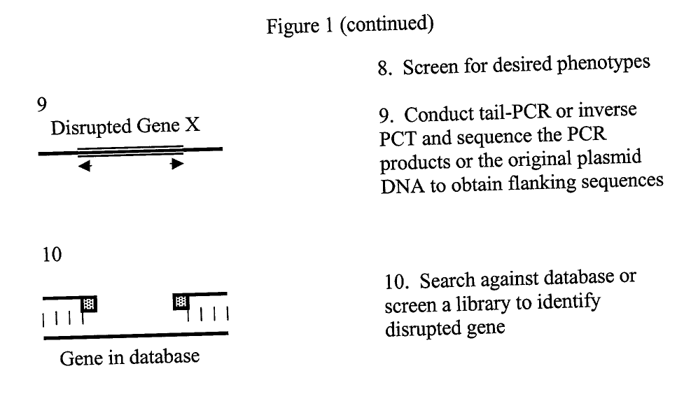

Knowledge of the fungal genes essential for life, and those controlling molecular mechanisms of pathogenicity, would suggest both fungicide targets and strategies by which plants resistant to disease might be developed. Toward this end, a genome-wide approach was used to identify such genes in Cochliobolus heterostrophus, a pathogen of maize (FIG. 1).

### Methods

### Generation of 10 kb genomic DNA fragments

Genomic DNA was isolated from C. heterostrophus wild type strain (C4 using the procedures described in Garber et al. (Anal. Biochem., 135: 416, 1983). The fungal genomic DNA was randomly sheared to about 10 kb using the Hydroshear machine. Sheared DNA fragments were end-filled using the Single dA Tailing Kit (Novagen). The adaptor:

| | |
|---|---|
| 5′ | CTTTAGAGCACA (SEQ ID NO. 2) |
| | ******** |
| 3′ | GAAATCTC |

was then added to the blunted genomic DNA fragments. DNA fragments of about 10 kb with adaptor were isolated from an 1% agarose gel and purified using QIAquick Gel Extraction Kit (QIAGEN).

### Construction of vectors

### pJWU1 construction

Plasmid pGEM-11 Zf(Promega) was digested with BamHI and ApaI, end-filled with DNA Polymerase I Large Fragment (Klenow, NEB), and then religated to generate plasmid pJWU1.

### pJWU3 construction

Plasmid pJWU1 was digested with XbaI, end-filled with Klenow, and then cut with SalI. This product was isolated from a 1% agarose gel and purified using QIAquick Gel Extraction Kit (QIAGEN). Plasmid pOT2A was digested with BglII, blunt ended with Klenow, and then cut with XhoI. The plasmid fragment (1 kb) containing the sacB gene with BstXI sites on each side was isolated and purified. This DNA fragment was ligated into XbaI blunt ended/SalI digested pJWU1 to yield plasmid pJWU3.

### Construction of a library with 10 kb genomic DNA inserts

pJWU3 was digested with BstXI and purified on a 1% agarose gel using QIAquick Gel Extraction Kit (QIAGEN). Gel isolated 10 kb genomic DNA fragments with BstXI adaptors were inserted into the purified vector to generate a library of 10 kb inserts.

### Construction of a library carrying a fungal selectable marker

The 10 kb DNA library was transformed into, and amplified in, E. coli strain DH5α Library DNA was isolated and digested with SalI, which does not cut the vector, but is expected to cut the insert DNA more than once. Digested DNA was dephoshorylated with Thermosensitive Alkaline Phosphatase (TsAP, GIBCOBRL). Plasmid pUCATPH (Lu et al., Proc. Natl. Acad. Sci. USA, 91:12649, 1994) containing the E. coli hygromycin B resistance gene hygB with the Aspergillus nidulans TRPC (Cullen et al., Gene, 57:21, 1987) promoter and terminator was digested with SalI. The fragment containing the hygB cassette (2.3 kb) was isolated (gel purification) and purified twice by QIAquick Gel Extraction Kit (QIAGEN). The purified hygB cassette fragment was then ligated to the SalI digested library DNA described above to create a second library. E. coli strain DH5α was used as a host for amplification of deletion library DNA.

Restriction enzyme digestion of miniprep DNA revealed that 95% of the constructs tested carried hygB and the size of fungal DNA replaced by hygB gene varied from 1.5 to 9.4 kb.

### Transformation of Cochliobolus heterostrophus protoplasts with the random deletion library DNA

A total of 50,000 colonies from the deletion library were picked individually and stored in microtiter dishes. The yield of plasmid DNA prepared using the GeneMachines robot is more than adequate for fungal transformation. Prior to transformation, each plasmid was digested with rare-cutting enzymes SfiI and NotI to release the insert carrying hygB plus fungal DNA remaining after hygB replacement. Each resulting linear DNA insert was transformed into C. heterostrophus protoplasts by conventional procedures (see, for example, Turgeon et al., Mol. Gen. Genet. 215:270, 1993).

### Identification and purification of transformants

Transformants are usually heterokaryons (mixture of wild type and transformed nuclei). Therefore, the transformed nuclei need to be isolated from wild type nuclei before phenotype of the deletion can be assayed. Formation of the vegetative spores (conidia) resolves nuclei. If 100% of the spores are hyg^{R}, then the transformant was a homokaryon with 100% transformed nuclei. If a transformant yields some hyg^{R} and some hyg^{S} spores, it is a heterokaryon and hyg^{R} conidia must be rescued. If 100% of the spores are hyg^{S}, the original transformant was a heterokaryon; all hyg^{R} nuclei must be dead. This class of transformants is one in which essential genes have been deleted.

For each transformation, two putative transformants were selected, assigned a number corresponding to the plasmid used for transformation, and transferred to complete, non-selective medium for conidiation and purification. In addition, a plug of each transformant was transferred to a fresh plate of selective medium (CMN Shyg; Lu et al., Proc. Natl. Acad. Sci. USA, 91:12649, 1994) to verify resistance to hygromycin B. When cultures have conidiated on nonselective medium, single conidia are streaked on CMNShyg so they are separated from each other, then single hyg^{R} conidia, are cut out after germination and transferred to a small CMNShyg plate. Two transformants (A and B) per plasmid transformation are purified by single conidiation and two purified hygR conidia from each stored in glycerol at −80° C.

### Determination of pathogenicity of deletion strains by plant tests

From each transformation, four purified strains were stored (two transformants, two purified conidia from each). Two strains (one from A, one from B) were tested on corn by spraying 1000 conidia/ml (15 mls) on 6 corn plants at the 4 leaf stage (one cotyledon, 2 leaves fully out, 4^{th} leaf just coming out). Plants were held at high humidity overnight, removed to room temperature and third leaves are scored at 3 and 4 days after inoculation. Lesion development was observed, recorded, and compared to wild type.

### Results

Transformants with essential genes deleted or altered virulence phenotypes were identified. For example, most primary transformants were heterokaryons, i.e., they contain both transformed and wild type nuclei. Routinely each transformant is genetically purified by isolating a single conidium, which resolves the heterokaryon, that contains the transformation selectable marker, e.g., E. coli gene (hygR) for resistance to the antibiotic hygromycin. If there are no conidia resistant to hygromycin from a particular transformant, the mutation in that transformant may be lethal, i.e., the primary transformant lives because the wild type nuclei rescue the dead transformed nuclei; a single conidium containing only transformed nuclei cannot grow because the mutation is in an essential gene.

To screen for virulence, each genetically purified transformant was grown in culture to produce conidia, the infective asexual spores. Conidia were suspended in water containing 0.01% detergent and sprayed on the foliage of 3 week old corn plants. The inoculated plants are incubated in a water saturated atmosphere for 16 hours, to keep the leaf surfaces wet, then held at 24EC with 16 hours light/day. Symptoms appear after 2 days, and were recorded at 3, 4, and 5 days. Mutants were identified by an altered pattern of disease development. To determine the sequences deleted in each, the plasmid used for transformation was used as a template for four sequencing reactions, two from the hygB selectable marker into the Cochliobolus DNA flanks and two from the vector into the Cochliobolus flanks. These data were employed to clone, amplify or otherwise isolate the corresponding non-deleted Cochliobolus genomic DNA (Tables 1-3).

TABLE 1

| | Amount | | | |
|---|---|---|---|---|
| Plasmid | Deleted (kb) | Strain | % hygB | Phenotype |
| pJWU4 | 8.2 | D.C4.4A1 | 67 | wt |
| pJWU5 | 9.4 | D.C4.5A2 | 80 | wt |
| pJWU6 | 2.6 | D.C4.6A1 | 91 | wt |
| pJWU7 | 1.4 | D.C4.7A1 | 87 | wt |
| pJWU8 | 5.6 | D.C4.8B2 | 6 | reduced |
| | | | | phathogenicity |
| pJWU9 | 1.4 | D.C4.9A1 | 3 | lethal |
| pJWU10 | 3.9 | D.C4.10A2 | 100 | wt |
| pJWU11 | 9.5 | D.C4.11B2 | 55 | reduced |
| | | | | pathogenicity |
| pJWU12 | 8.6 | D.C4.12A2 | 6 | lethal |
| pJWU13 | 1.8 | D.C4.13A1 | 100 | wt |
| pJWU15 | 7.1 | D.C4.15A1 | 100 | wt |
| pJWU16 | 3.5 | D.C4.16A1 | 100 | wt |
| pJWU17 | 7.4 | D.C4.17A1 | 100 | wt |
| pJWU18 | 6.5 | D.C4.18A1 | 97 | wt |
| pJWU19 | 4.4 | D.C4.19A1 | 35 | wt |
| pJWU20 | 8.9 | D.C4.20A1 | 6 | conidium |
| | | | | germination |
| | | | | lethal |
| pJWU21 | 6.7 | D.C4.21B1 | 8 | lethal |

TABLE 2
Plasmids with Random Deletion

Query of Database

| Plasmid | Strain | Primer 1 | Primer 2 |
|---|---|---|---|
| pJWU-4 | | none** | contig9515 |
| | D.C.4.4 | | |
| pJWU-5 | D.C.4.5 | contig6317 | contig8299 |
| pJWU-6 | D.C.4.6 | contig5808 | contig6847 |
| pJWU-7 | D.C.4.7 | none | contig7584 |
| pJWU-8 | D.C.4.8 | contig8709 | contig5865 |
| pJWU-9 | D.C.4.9 | contig9579 | contig9579 |
| pJWU-10 | D.C.4.10 | contig9591 | contig9591 |
| pJWU-11 | D.C.4.11 | none | none |
| pJWU-12 | D.C.4.12 | contig8299 | contig8299 |
| pJWU-13 | D.C.4.13 | contig4731 | contig7584 |
| pJWU-15 | D.C.4.15 | contig8237 | none |
| pJWU-16 | D.C.4.16 | contig9579 | contig397 |
| pJWU-17 | D.C.4.17 | contig4231 | contig4231 |
| pJWU-18 | D.C.4.18 | contig5437 | none |
| pJWU-19 | D.C.4.19 | contig7421 | contig7421 |
| pJWU-20 | D.C.4.20 | none | none |
| pJWU-21 | D.C.4.21 | contig5191 | contig6317 |
| pJWU-22 | D.C.4.22 | none | none |

TABLE 3

| | Approx | | | | |
|---|---|---|---|---|---|
| | amount | | | | |
| | DNA | | Percent | | |
| | deleted | | hygR | | Related to |
| Plasmid | (kb) | Strain | conidia* | Phenotype | contig |
| pJWU-8 | 5.8 | D.C.4.8.B2 | 8 | reduced | co5contig8709, |
| | | | | virulence | 5865 |
| pJWU-9 | 1.8 | D.C4.9C | 3 | lethal | co5contig9579 |

The method of the invention can be employed with DNA and cells from other organisms, including other filamentous fungi, plants, microorganisms, and vertebrates. In particular, the method is useful for deletion analyses in undifferentiated cells such as mammalian stem cells.

In addition, to allow for deleted transformants to be processed in pools, bar codes may be added to the vector in which the deletion library is prepared. For example, it might be possible to inoculate plants with pools of transformants. Bar codes that cannot be recovered are evidence for genes associated with of virulence.

The method of the invention is also useful for directed or targeted gene deletions. For example, genes for secondary metabolism (e.g., peptide synthetases) may be required for pathogenicity. A plasmid having a deleted peptide synthetase gene is introduced to the corresponding wild type cell. A homologous recombinant is then tested for its pathogenicity on a susceptible host.

### Example 2

DNA adjacent to the marker gene was sequenced using primers that annealed to the 5′ and 3′ ends of the marker gene. In addition, Cochliobolus DNA adjacent to vector sequences in the plasmid employed for transformation was sequenced using primers that annealed to the vector sequences 5′ and 3′ to the inserted Cochliobolus DNA. The sequence data obtained from these sequencing reactions was compared to contigs from a Cochliobolus sequence database and open reading frames in the corresponding contig were determined.

For example, one mutant, designated D.C4.8B2, displayed low virulence when tested on plants. The Cochliobolus DNA in the plasmid used to prepare the mutant, pJWU8, was sequenced and those sequences corresponded to DNA in co6contig8709 and co6contig5865. Contig 8709-5865 (SEQ ID NO.3) was found to contain open reading frames corresponding to the deleted sequence. This analysis also showed that the plasmid had a 5.8 Kb deletion in genomic DNA sequences. Four open reading frames (designated ORF-1 through ORF-4) were identified. ORF-1 (SEQ ID NO.7, SEQ ID NO:8) encodes a 647 amino acid polypeptide having a molecular weight of approximately 71,463 daltons, ORF-2 (SEQ ID NO.9, SEQ ID NO.10) encodes a 211 amino acid polypeptide having a molecular weight of about 23,104 daltons, ORF-3 (SEQ ID NO.11, SEQ ID NO.12) encodes a 754 amino acid polypeptide having a molecular weight of approximately 84,075 daltons, and ORF-4 (SEQ ID NO.13, SEQ ID NO.14) encodes a 339 amino acid polypeptide having a molecular weight of about 35,487 daltons. To determine the function of the gene product encoded by each ORF, BLAST searches were conducted. The gene product encoded by ORF-1 is structurally related to the aryl-alcohol oxidase precursor from Pleurotus enyngii and to the versicolorin B synthase from Aspergillus parasiticus (Silva et al., J. Biol. Chem., 271:13600, 1996; McGuire et al., Biochemistry, 35:11470, 1996; Watanabe et al., Chem. Biol., 3:463, 1996; Silva et al., J. Biol. Chem., 272:804, 1997). The gene product of ORF-2 is structurally related to the NTP pyrophosphohydrolase from Streptomyces coelicolor, and the gene product of ORF-3 is structurally related to cytochrome P450 from rat and other organisms. The function for the gene product of ORF-4 is unknown. BLAST searches also provided potential orthologs of the gene products.

Another mutant, D.C4.9, displayed a lethal phenotype, indicting the deletion of an essential gene. A similar analysis to the for D.C4.8B2, demonstrated that the sequences in D.C4.9 were related to those in co6ocontig9092 and that the corresponding plasmid had a 1.8 kb deletion in genomic Cochliobolus DNA. A single ORF (SEQ ID NO.5, SEQ ID NO.6) was found in contig 9092 (SEQ ID NO.1). The open reading frame encodes a 2698 amino acid polypeptide having a molecular weight of approximately 305,910 daltons. The polypeptide is highly related to the YHR099W protein, the TRRAP-like protein from yeast, and the TRRAP protein from human (see WO 98/50550). In addition, a 2 kb region upstream of each gene contains the promoter region for each of the 5 genes (SEQ ID NOs.15-19).

### Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for preparing a library of modified DNA fragments comprising:
contacting a library of DNA fragments in a vector with an agent so as to cause at least one double strand break in at least one fragment to yield a library of DNA fragments having at least one double strand break; and
inserting a detectable polynucleotide or gene into the break so as to yield a library of modified DNA fragments.

2. The method of claim 1, wherein said DNA is selected from the group consisting of plant DNA, fungal DNA, avian DNA, and mammalian DNA.

3. The method claim 1, wherein said vector is selected from the group consisting of a plasmid, a phage, a bacterial artificial chromosome, a yeast artificial chromosome and a cosmid.

4. The method of claim 1, wherein said detectable nucleotide sequence or gene comprises a selectable marker or a screenable marker.

5. The method of claim 1, wherein said library of DNA fragments is contacted with at least one endonuclease.

6. The method of claim 5, wherein said at least one endonuclease does not have a recognition site in said vector, but has at least one recognition site in at least one DNA fragment.

7. The method of claim 1, wherein said library is a cDNA library or a genomic library.

8. A library prepared by the method of claim 1.

9. A method for identifying the function of a gene comprising:
contacting cells with the library of claim 8 so as to yield a population of cells containing at least one recombinant cell, in which homologous recombination has occurred between the genome of the cell and the modified DNA in at least one member of the library; and
identifying the recombinant cell by a change in phenotype.

10. The method of claim 9, wherein said recombinant cell is selected from the group consisting of plant cells, bacterial cells, fungal cells, avian cells, and mammalian cells.

11. An organism comprising at least one cell of claim 10.

12. An isolated polynucleotide comprising a nucleotide sequence selected from the group consisting of:
a) any one of SEQ ID NO.1, SEQ ID NO.3, SEQ ID NO.6, SEQ ID NO.8, SEQ ID NO.10, SEQ ID NO.12, SEQ ID NO.14,
b) the complement of any of the sequences of a,
c) a sequence substantially similar to any of the sequences of a, and
d) the complement of any of the sequences of c.

13. An isolated polypeptide comprising any one of SEQ ID 5, SEQ ID NO.7, SEQ ID NO.9, SEQ ID NO.11 and SEQ ID NO.13.

14. An isolated polynucleotide comprising a nucleotide sequence encoding any one of the polypeptides of claim 13.

15. An isolated polypeptide comprising an amino acid sequence substantially similar to any one of SEQ ID 5, SEQ ID NO.7, SEQ ID NO.9, SEQ ID NO.11 and SEQ ID NO.13.

16. An isolated polynucleotide comprising a nucleotide sequence encoding any one of the polypeptides of claim 15.

17. An expression cassette comprising as operably linked components, a promoter and an isolated polynucleotide of claim 12.

18. A recombinant vector comprising the expression cassette of claim 17.

19. A host cell comprising the recombinant vector of claim 18.

20. The host cell of claim 19, wherein said host cell is selected from the group consisting of bacterial cells, yeast cells, fungal cells, plant cells, and animal cells.

21. An organism comprising a host cell of claim 20.

22. A method for identifying an agent having anti-fungal activity comprising, contacting a fungus with an agent; determining if the agent binds to at least one of the polypeptides of claim 13; and determining the effect of said binding on fungal viability.

23. An agent identified by the method of claim 22.

24. A method for identifying an agent having anti-fungal activity comprising, contacting a fungus with an agent; determining if the agent binds to at least one of the polypeptides of claim 15; and determining the effect of said binding on fungal viability.

25. An agent identified by the method of claim 24.

26. An isolated polynucleotide comprising a regulatory region having a sequence selected from the group consisting of SEQ ID NO.15, SEQ ID NO.16, SEQ ID NO.17, SEQ ID NO.18 and SEQ ID NO.19.

27. A fragment of the isolated polynucleotide of claim 26, wherein said fragment comprises a minimal promoter.

28. An isolated polynucleotide comprising a regulatory region having a sequence substantially similar to a sequence selected from the group consisting of SEQ ID NO.15, SEQ ID NO.16, SEQ ID NO.17, SEQ ID NO.18 and SEQ ID NO.19.

29. A fragment of the isolated polynucleotide of claim 28, wherein said fragment comprises a minimal promoter
